(19) United States
(12) Patent Application Publication
(10) Pub. No.: US 2023/0407365 A1
(43) Pub. Date: Dec. 21, 2023

(54) DIAGNOSTIC TOOL FOR SCREENING THE IMPACT OF PRODUCT INGREDIENTS FOR UROGENITAL MICROBIOMES THERAPIES

(71) Applicants: Pamela Miles, Boston, MA (US); Cindy Orser, Boulder, CO (US)

(72) Inventors: Pamela Miles, Boston, MA (US); Cindy Orser, Boulder, CO (US)

(21) Appl. No.: 17/901,826

(22) Filed: Sep. 1, 2022

Related U.S. Application Data
(60) Provisional application No. 63/354,232, filed on Jun. 21, 2022.

(51) Int. Cl. C12Q 1/18; G01N 33/15

(52) U.S. Cl. CPC C12Q 1/18; G01N 33/15; G01N 2333/335

(57) ABSTRACT

The embodiments disclose a method including determining populations of Lactobacilli bacterial species associated with a predetermined health level for genital microbiome conditions to screen ointment preparations and ingredients for their impact on the well-being of said bacterial species, identifying the dominant Lactobacillus species in the predetermined health level for genital microbiome conditions, gathering culturing data and monitoring data of different optional cannabinoid or terpenoid compounds on genital Lactobacilli cultures, analyzing the culturing data and monitoring data to assay categorized impact data of the different optional cannabinoid or terpenoid compounds on the genital Lactobacilli cultures, and providing a diagnostic tool for screening the impact of a ointment preparations and ingredients on the genital community microbiomes for the design of safe, effective ointment preparations and ingredients for genital use.

### CROSS-REFERENCED TO RELATED APPLICATIONS

This Patent Application is based on U.S. Provisional Patent Application Ser. No. 63/354,232 filed Jun. 21, 2022, entitled “ANTIMICROBIAL EFFECTS OF CANNABINOIDS ON HUMAN UROGENITAL BIOME IN VITRO SCREENING ASSAY METHOD AND DEVICES”, by Pamela Miles et al., the U.S. Patent Application being incorporated herein by reference.

### BACKGROUND

One of the common conditions experienced by women throughout their lives is vaginitis, typically characterized in the medical field as an inflammation of the vagina that can result in discharge, itching, and pain. There are several potential etiologies of vaginitis, including candida vaginitis, typically caused by overgrowth with the commensal fungal organism Candida Albicans, trichomonas vaginitis, which is a sexually transmitted infection (STI) caused by a protozoan parasite, Trichomonas urogenitalis, vaginal atrophy, or atrophic vaginitis, which results from reduced estrogen levels during menopause, and bacterial vaginosis or vaginitis, which is associated with a perturbation in the composition of the bacterial microflora of the vagina. Vaginitis symptoms may include a change in color, odor, or amount of discharge from a woman's vagina, genital itching or irritation, pain during intercourse, painful urination, and light vaginal bleeding or spotting. Vaginitis symptoms can lead to various degrees of physical and emotional discomfort and lower the overall quality of life. Vaginitis symptoms can also be a sign of an underlying infection, which should be promptly identified and treated in order to avoid medical complications, and, in case of an STI, to avoid further transmission.

Cannabinoids are a class of chemicals that can act on endocannabinoid receptors and have been explored recently in addressing reproductive health conditions owing to their analgesic properties. Cannabinoids have been utilized in treatments for addressing conditions including, but not limited to, pelvic floor dysfunction, dysmenorrhea, vulvodynia, vaginosis, endometriosis, and dyspareunia. Well-known cannabinoids include tetrahydrocannabinolic acid (THCA), delta-9-tetrahydrocannabinol (THC), cannabidiolic acid (CBDA), cannabidiol (CBD), and cannabigerol (CBG). At least 85 different cannabinoids have been isolated from cannabis. Cannabinoid receptor ligands include endocannabinoids, which can be found naturally occurring in humans and other animals.

Currently, cannabinoid compositions are considered to have a wide scope of therapeutic applications. Current cannabinoid compositions are delivered through combustion smoking, vaping, orally, topically, and vaginally. Current cannabinoid composition delivery mechanisms result in the failure of the cannabinoid compositions in reaching their intended target region with efficacious doses as cannabinoids do not act systemically.

The human vagina has been explored as a direct route of cannabinoid delivery due to its potential as a non-invasive route of drug administration as well as the presence of a dense network of blood vessels for both systemic and local effect. The main advantages of vaginal drug delivery over conventional drug delivery are the ability to by-pass first-pass metabolism in the liver, ease of administration, and high permeability for low molecular weight drugs. To assess the viability of the vagina as the location of cannabinoid drug administration, there remains a substantial need for a method to assess the impact of cannabinoids on the microbiome of the human vagina.

### SUMMARY

The embodiments described herein present a method for studying antimicrobial and antifungal effects of cannabinoids as well as other ingredients and intimate care products on the human vaginal microbiome types, including the steps of culturing one or more microbes, adding one or more cannabinoid compounds to the microbe culture, measuring a pH level of the microbe culture treated with the one or more cannabinoid compounds, determining a lactic acid level the microbe culture treated with the one or more cannabinoid or other product ingredients, and analyzing the impact on the population count of the specific microbial culture based on qPCR data.

### BRIEF SUMMARY OF THE INVENTION

The present invention provides the ability to categorize products and their ingredients based on their impact on the key Lactobacilli and urogenital community microbiota type species associated with healthy urogenital microbiomes. The invention can either be used qualitatively to derive an “inhibitory” or “non-inhibitory” outcome or it can be used quantitatively through an additional qPCR step to provide the degree of inhibition. The invention can be used to inform the choice of available therapies based on favorable screening outcomes against the key community microbiomes as well as the inhibition of invasive pathogens and opportunistic microbes. The terms “invention,” “the invention,” “this invention,” and “the present invention,” used in this patent are intended to refer broadly to all of the subject matter of this patent and the patent claims below. Statements containing these terms should be understood not to limit the subject matter described herein or to limit the meaning or scope of the patent claims below. Covered by the patent embodiments of the invention are defined by the claims, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter.

### FIELD OF THE INVENTION

The disclosure relates to a method and system of studying the antimicrobial and antifungal effects of cannabinoids on microbes commonly represented in the healthy urogenital ecosystem. More particularly, the disclosure relates to a method and system of studying the antimicrobial and antifungal effects of cannabinoids on Lactobacilli populations in the urogenital ecosystem, and the antifungal effect of cannabinoids on common microbes and invasive pathogens in the urogenital system.

### BRIEF DESCRIPTION OF THE DRAWINGS

FIG. 1 shows for illustrative purposes only an example of an overview of categorized products and their ingredients based on their impact on a healthy urogenital microbiome of one embodiment.

FIG. 2 shows for illustrative purposes only an example of antimicrobial effects of cannabinoids on microbes of one embodiment.

FIG. 3 shows for illustrative purposes only an example of levels of different Lactobacillus bacterial populations of one embodiment.

FIG. 4 shows for illustrative purposes only an example of a urogenital microbiome assay network platform of one embodiment.

FIG. 5 shows for illustrative purposes only an example of the impact of CBD, THC, and CBD/THC combination of one embodiment.

FIG. 6 shows for illustrative purposes only an example of a CBD treatment on anaerobically cultured Lactobacillus crispatus populations of one embodiment.

FIG. 7 shows for illustrative purposes only an example of the impact of THC and other cannabinoids on anaerobically cultured Lactobacillus gasseri and Lactobacillus jensenii populations of one embodiment.

FIG. 8 shows for illustrative purposes only an example of non-cannabinoid parameter adjustments of one embodiment.

FIG. 9 shows for illustrative purposes only an example of depicting an overview of a cell culture and analysis procedure of one embodiment.

FIG. 10 shows for illustrative purposes only an example of pure Lactobacillus crispatus cultures grown in a Lactobacillus selective media of one embodiment.

FIG. 11 shows for illustrative purposes only an example of quantitating a personal care product's microbiome impact results of one embodiment.

FIG. 12 shows for illustrative purposes only an example of the urogenital microbiome assay analysis application of one embodiment.

FIG. 13 shows for illustrative purposes only an example of adjusting assay parameters of one embodiment.

FIG. 14 shows for illustrative purposes only an example of in vitro screening assay product's toxic microbiome impact results of one embodiment.

FIG. 15 shows for illustrative purposes only an example of the impact results for products based on the demographic differences of one embodiment.

FIG. 16A shows for illustrative purposes only an example of studying CBD and other cannabinoids on anaerobically cultured Candida albicans of one embodiment.

FIG. 16B shows for illustrative purposes only an example of water-soluble CBD effects on C. albicans of one embodiment.

FIG. 17A shows for illustrative purposes only an example of the results for Lactobacillus crispatus of one embodiment.

FIG. 17B shows for illustrative purposes only an example of the results for Lactobacillus gasseri of one embodiment.

FIG. 18 shows for illustrative purposes only an example of the results for E. coli of one embodiment.

FIG. 19 shows for illustrative purposes only an example of L. crispatus in Anaerobic Conditions at 37° C. of one embodiment.

FIG. 20 shows for illustrative purposes only an example of L. gasseri in Anaerobic Conditions at 37° C. of one embodiment.

FIG. 21 shows for illustrative purposes only an example of inhibition results of one embodiment.

FIG. 22 shows for illustrative purposes only an example of L. gasseri in Anaerobic Conditions at 30° C. of one embodiment.

FIG. 23 shows for illustrative purposes only an example of results of cultures of Lactobacilli jensenii exposed to three different CBD products of one embodiment.

FIG. 24 shows for illustrative purposes only an example of commercial products tested results of one embodiment.

FIG. 25 shows for illustrative purposes only an example of normalized CT values to control of one embodiment.

FIG. 26 shows for illustrative purposes only an example of control normalized at log10 CFU/mL of one embodiment.

FIG. 27 shows for illustrative purposes only an example of Exposure to CBD Product A of one embodiment.

FIG. 28 shows for illustrative purposes only an example of Exposure to CBD/THC Product B of one embodiment.

FIG. 29 shows for illustrative purposes only an example of a body wash exposure study of one embodiment.

### DETAILED DESCRIPTION OF THE INVENTION

In a following description, reference is made to the accompanying drawings, which form a part hereof, and in which is shown by way of illustration a specific example in which the invention may be practiced. It is to be understood that other embodiments may be utilized, and structural changes may be made without departing from the scope of the present invention.

### General Overview

It should be noted that the descriptions that follow, for example, in terms of antimicrobial effects of cannabinoids on the human urogenital microbiome in vitro screening assay method and devices are described for illustrative purposes and the underlying system can apply to any number and multiple types of microbiomes. In one embodiment of the present invention, the antimicrobial effects of cannabinoids on human urogenital microbiome in vitro screening assay method and devices can be configured using CBD. The antimicrobial effects of cannabinoids on human urogenital microbiome in vitro screening assay method and devices can be configured to include biome microbes cultured aerobically and can be configured to include biome microbes anaerobically using the present invention.

New laboratory research has demonstrated that some intimate care products currently on the market can significantly inhibit the growth of lactobacilli, which are essential bacteria for a healthy vagina. The compositions of bacteria which coat the walls of the vagina (often called the vaginal microbiome or VMB) are crucial to maintaining a healthy pH and preventing infections. The dominance of lactobacilli in the vaginal microbiome makes for the healthiest and most resilient condition to protect against infection.

The results raise concerns that urogenital exposure from the use of some intimate care products could adversely affect the lactobacilli balance. A lack of good lactobacilli balance can lead to significant health problems including bacterial vaginosis (BV), increased risk of sexually transmitted diseases, and fertility concerns. Of particular concern, is the impact some of the tested products have on Lactobacillus iners, the dominant Lactobacillus species in the vaginal microbiome of Black women. Due in part to targeted marketing tactics, Black women in the U.S. are more likely to use intimate care products than women of other races.

Due to a lack of regulation, manufacturers are not required to test a product's impact on the urogenital microbiome. Nor are they required to meet any universal standards of ingredient safety. This preliminary testing indicates that manufacturers need to take more responsibility for the impacts these products are having on people's health. Regulation is needed to require manufacturers to test intimate care products on the urogenital microbiome. There is a wide variety of intimate care products on the market, and because of a lack of testing and publicly available data, we do not know which other products could be having this same effect. More research and testing of products are needed to assure that intimate care products are not harming our health. The following descriptions are of the in vitro assay to evaluate the anti-microbial activity of eleven intimate care products. (The assay was done in the laboratory in test tubes and petri dishes and did not involve human or animal testing.) The products tested included lubricants, genital moisturizers, washes, deodorants, and vaginal suppositories, as well as cannabidiol (CBD) isolate and tetrahydrocannabidiol (THC) isolate since several of the products also included one or both cannabinoids. Specifically, the testing looked at whether products inhibited the growth of individual species of urogenital lactobacilli including Lactobacillus crispatus, Lactobacillus gasseri, Lactobacillus iners, and Lactobacillus jensenii. Products tested included both popular brands that have been on the market for a long time, as well as newer products.

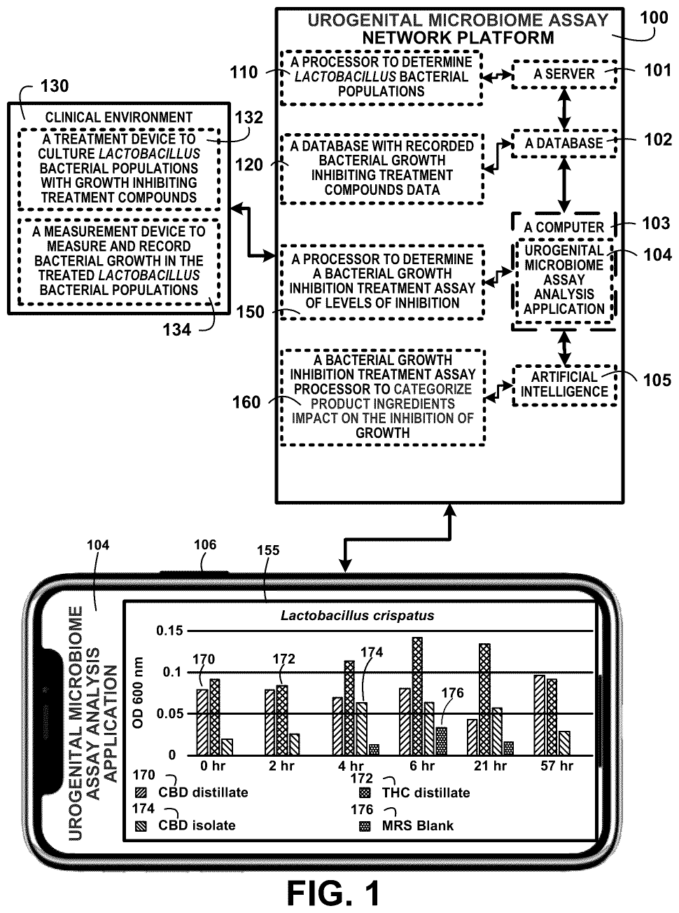

FIG. 1 shows for illustrative purposes only an example of an overview of categorized products and their ingredients based on their impact on a healthy urogenital microbiome of one embodiment. FIG. 1 shows a urogenital microbiome assay network platform 100 including a server 101, a database 102, a computer 103 with a urogenital microbiome assay analysis application 104, and an artificial intelligence 105 system. The urogenital microbiome assay network platform 100 includes a processor to determine Lactobacillus bacterial populations 110. A platform database with recorded bacterial growth inhibiting treatment compounds 120. In a clinical environment 130, growth inhibition is made with a treatment device to culture Lactobacillus bacterial populations with growth-inhibiting treatment compounds 132. A measurement device to measure and record bacterial growth in the treated Lactobacillus bacterial populations 134. The recordation of the bacterial growth measurements is on the database 102. A processor to determine a bacterial growth inhibition treatment assay of levels of inhibition 150. A bacterial growth inhibition treatment assay processor to categorize product ingredients impact on the inhibition of growth 160. The categorization process results are reported in an in vitro screening assay for quantitating an intimate care product's impact on the urogenital microbiome. The bacterial growth inhibition treatment assay showing growth measurements is transmitted to a user smart phone 106 with the urogenital microbiome assay analysis application 104. For example, Lactobacillus crispatus growth inhibition measurements 155 where the Lactobacillus crispatus has been treated with CBD distillate 170, THC distillate 172, CBD isolate 174, and MRS blank 176 of one embodiment.

### Detailed Description

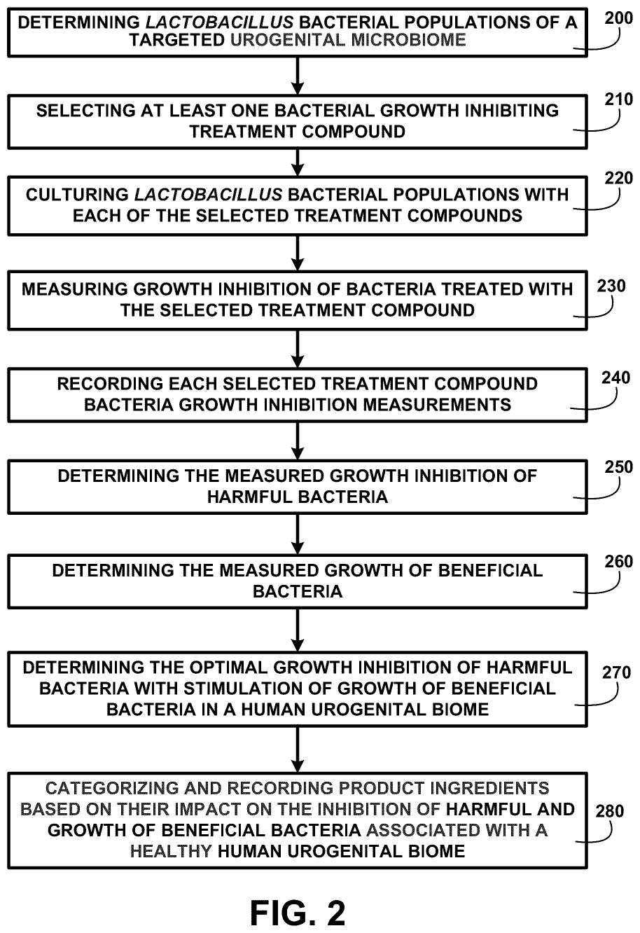

FIG. 2 shows for illustrative purposes only an example of antimicrobial effects of cannabinoids on microbes of one embodiment. FIG. 2 shows the antimicrobial effects of cannabinoids on microbe's representative of the human urogenital biome, notably Lactobacillus species. The process includes determining Lactobacillus bacterial populations of a targeted urogenital microbiome 200. Selecting at least one bacterial growth inhibiting treatment compound 210. Culturing Lactobacillus bacterial populations with each of the selected treatment compounds 220. Measuring growth inhibition of bacteria treated with the selected treatment compound 230. Recording each selected treatment compound bacteria growth inhibition measurements 240. Determining the measured growth inhibition of harmful bacteria 250. Determining the measured growth of beneficial bacteria 260. Determining the optimal growth inhibition of harmful bacteria with stimulation of growth of beneficial bacteria in a human urogenital biome 270. Categorizing and recording product ingredients based on their impact on the inhibition of harmful and growth of beneficial bacteria associated with a healthy human urogenital biome 280 of one embodiment.

The cell culture and analysis procedure includes cannabinoid compounds and natural or synthetic molecules with basic cannabinoid structures that are synthetically modified to provide cannabinoid analogs. Culturing representative urogenital biome microbes can be cultured aerobically or anaerobically, particularly in a microaerophilic environment. Monitoring the cannabinoid compound treated Lactobacilli cultures with pH measuring apparatus and lactic acid level measuring apparatus, including using a high-performance liquid chromatography-ultraviolet (HPLC-UV) lactic acid level detector. The collective culturing data and monitoring data is used for analyzing the impact of the different cannabinoid compounds on the urogenital Lactobacilli cultures using a quantitative polymerase chain reaction (qPCR) analyzer of one embodiment.

### Levels of Different Lactobacillus Bacterial Populations

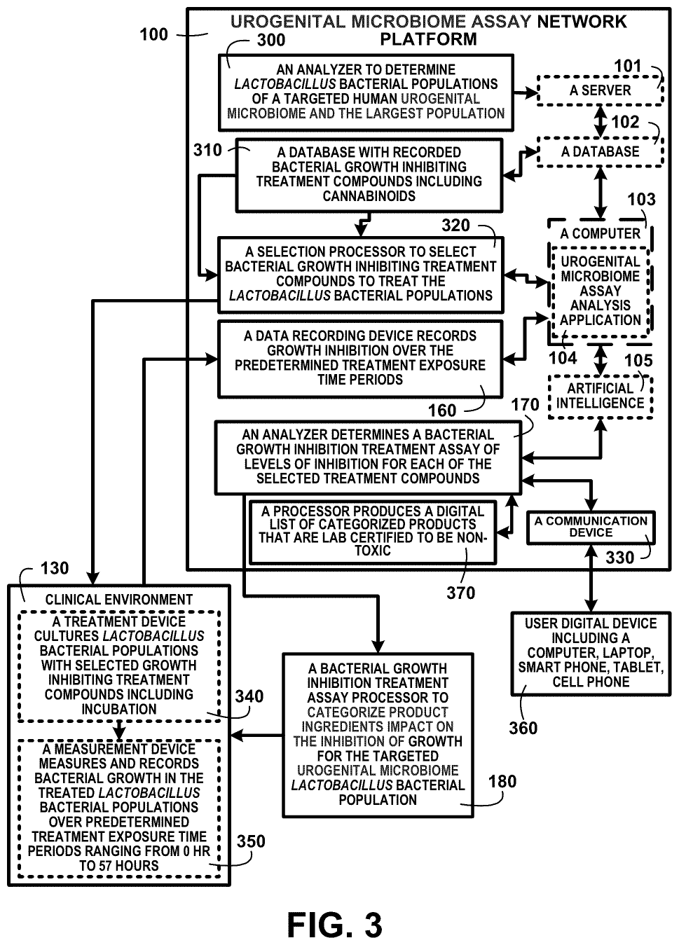

FIG. 3 shows for illustrative purposes only an example of levels of different Lactobacillus bacterial populations of one embodiment. FIG. 3 shows the urogenital microbiome assay network platform 100 including the server 101, database 102, computer 103, urogenital microbiome assay analysis application 104, and artificial intelligence 105. An analyzer to determine Lactobacillus bacterial populations of a targeted human urogenital microbiome and the largest population 300. A database with recorded bacterial growth inhibiting treatment compounds including cannabinoids 310. A selection processor to select bacterial growth inhibiting treatment compounds to treat the Lactobacillus bacterial populations 320. The different Lactobacillus bacterial populations and the selected treatment compounds are processed in a clinical environment 130. A treatment device cultures Lactobacillus bacterial populations with selected growth inhibiting treatment compounds including incubation 340. A measurement device measures and records bacterial growth in the treated Lactobacillus bacterial populations over predetermined treatment exposure time periods ranging from 0 hr. to 57 hours 350.

A data recording device records growth inhibition over the predetermined treatment exposure time periods 160. An analyzer determines a bacterial growth inhibition treatment assay of levels of inhibition for each of the selected treatment compounds 170. A bacterial growth inhibition treatment assay processor categorizes product ingredients impact on the inhibition of growth for the targeted urogenital microbiome Lactobacillus bacterial population 180. Product ingredients are processed in the clinical environment 130. A processor produces a digital list of categorized products that are lab certified to be non-toxic 370. A communication device 330 transmits the measurement data, and a digital list of categorized products to a user digital device including a computer, laptop, smart phone, tablet, or cell phone 360.

Levels of different Lactobacillus bacterial populations in a human urogenital microbionne, for example, the vagina of 4 healthy human subjects over a period of time. 33 subjects were looked at over a 4-month period, and participants self-collected urogenital swabs for 7 or 14 days, then again at 2 and 3 weeks to determine the urogenital biome impact of cannabinoid compounds, the dominant urogenital biome species was first identified with the swabs. Samples were then extracted for DNA using a DNA kit and assessed for bacterial 16S rRNA genes, then run against qPCR assays targeting multiple key urogenital bacteria associated with health and disease state to determine the majority population of the Lactobacilli species. Various Lactobacilli species from 4 healthy human subjects were monitored to identify the dominant Lactobacillus species over a period of approximately 4 weeks, quantified with a qPCR analysis that was carried out with the qPCR apparatus. Based on the outcome, the Lactobacillus crispatus bacterial population was chosen as a representative urogenital biome microorganism.

The human vagina in the majority of reproductive age women hosts an extensive bacterial population including, but not limited to, Lactobacillus crispatus, Lactobacillus iners, Lactobacillus gasseri, and Lactobacillus jensenii. Lactobacilli utilize glycogen breakdown products, such as maltose, to produce lactic acid. This acidification of the vagina to a pH of 3.0-4.5 results in the inhibition of the growth of other bacteria. Lactobacilli also bind to the surface of vaginal epithelial cells and compete with other microorganisms to prevent them from attaching to and infecting these cells. Lactobacilli further release soluble components that inhibit other bacteria from associating with the epithelial cell membrane. The production by Lactobacilli of compounds called bacteriocins, which kill other bacteria, also contributes to their dominance in the human vagina. Thus, maintaining a sustainable and stable Lactobacilli population, without sharp increases or decreases, in the human vagina is crucial for optimal health including but not limited to reproductive health, sexual function, and overall physical and mental health.

### Vaginal Microbiome Assay Network Platform

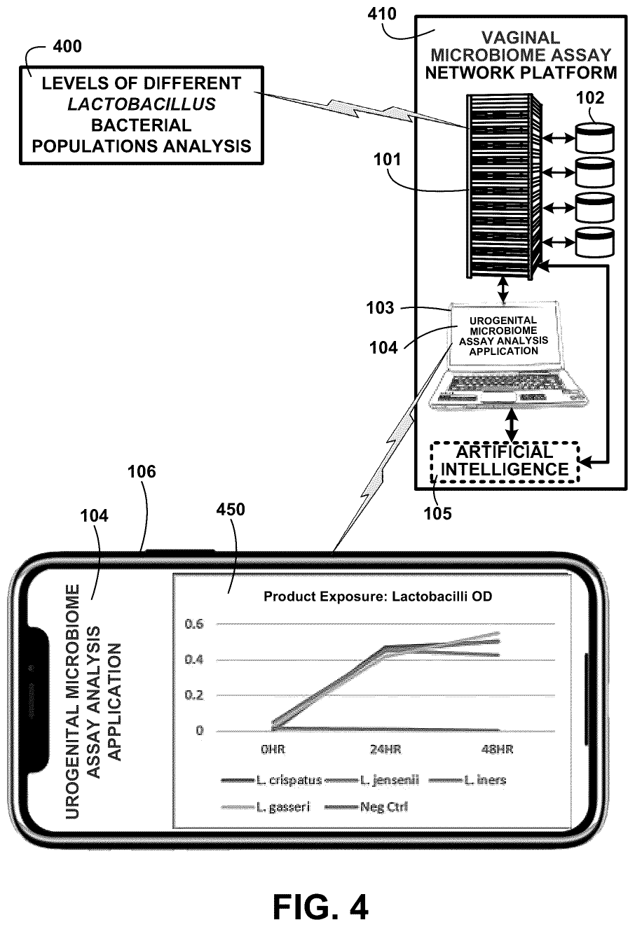

FIG. 4 shows for illustrative purposes only an example of a vaginal microbiome assay network platform of one embodiment. FIG. 4 shows levels of different Lactobacillus bacterial populations analysis 400 using product exposure. Wherein the results of the different Lactobacillus bacterial populations analysis are transmitted to a vaginal microbiome assay network platform 410. The vaginal microbiome assay network platform 410 includes a plurality of servers 101, a plurality of databases 102, a network computer 103 with a urogenital microbiome assay analysis application 104, and an artificial intelligence 105 device system. A graphic chart representation of the shows levels of different Lactobacillus bacterial populations analysis 400 under product exposure are transmitted to a user digital device 106 with the urogenital microbiome assay analysis application 104 as shown in an example of Lactobacilli exposure results 450 of one embodiment.

### Impact of CBD, THC, and CBD/THC Combination

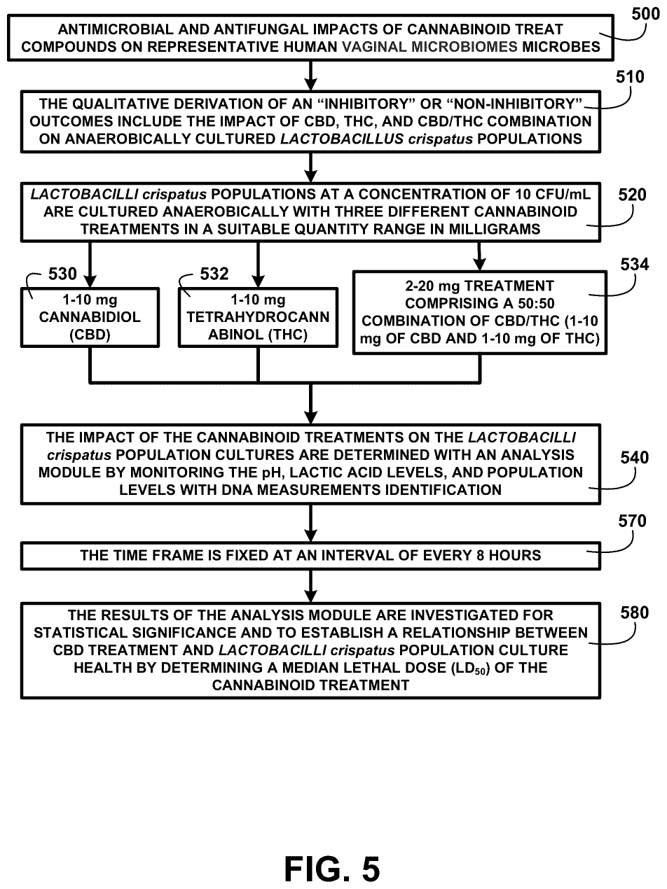

FIG. 5 shows for illustrative purposes only an example of the impact of CBD, THC, and CBD/THC combination of one embodiment. FIG. 5 shows antimicrobial and antifungal impacts of cannabinoid treat compounds on representative human vaginal microbiomes microbes 500. The qualitative derivation of an “inhibitory” or “non-inhibitory” outcomes include the impact of CBD, THC, and CBD/THC combination on anaerobically cultured Lactobacillus crispatus populations 510. Lactobacilli crispatus populations at a concentration of 10 CFU/mL are cultured anaerobically with three different cannabinoid treatments 520 in a suitable quantity range in milligrams. In one embodiment, a suitable quantity range in milligrams including 1-10 mg Cannabidiol (CBD) 530, and in other embodiments a lesser or greater range of cannabidiol (CBD), 1-10 mg Tetrahydrocannabinol (THC) 532, and 2-20 mg treatment comprising a 50:50 combination of CBD/THC (1-10 mg OF CBD and 1-10 mg of THC) 534. The impact of the cannabinoid treatments on the Lactobacilli crispatus population cultures are determined with an analysis module by monitoring the pH, lactic acid levels, and population levels with DNA measurements identification 540. The time frame is fixed at an interval of every 8 hours 570. The results of the analysis module are investigated for statistical significance and to establish a relationship between CBD treatment and Lactobacilli crispatus population culture health by determining a median lethal dose (LD₅₀) of the cannabinoid treatment 580 of one embodiment.

### A CBD Treatment on Anaerobically Cultured Lactobacillus crispatus Populations

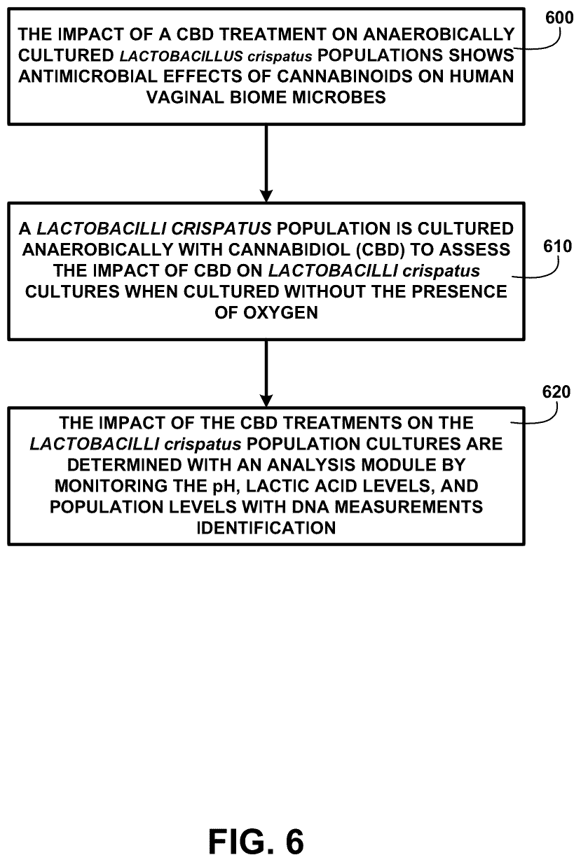

FIG. 6 shows for illustrative purposes only an example of a CBD treatment on anaerobically cultured Lactobacillus crispatus populations of one embodiment. FIG. 6 shows the impact of a CBD treatment on anaerobically cultured Lactobacillus crispatus populations and shows antimicrobial effects of cannabinoids on human vaginal biome microbes 600. A Lactobacilli crispatus population is cultured anaerobically with cannabidiol (CBD) to assess the impact of CBD on Lactobacilli crispatus cultures when cultured without the presence of oxygen 610. The impact of the CBD treatments on the Lactobacilli crispatus population cultures are determined with an analysis module by monitoring the pH, lactic acid levels, and population levels with DNA measurements identification 620 of one embodiment.

### Impact of THC and Other Cannabinoids on Anaerobically Cultured Lactobacillus gasseri and Lactobacillus jensenii Populations

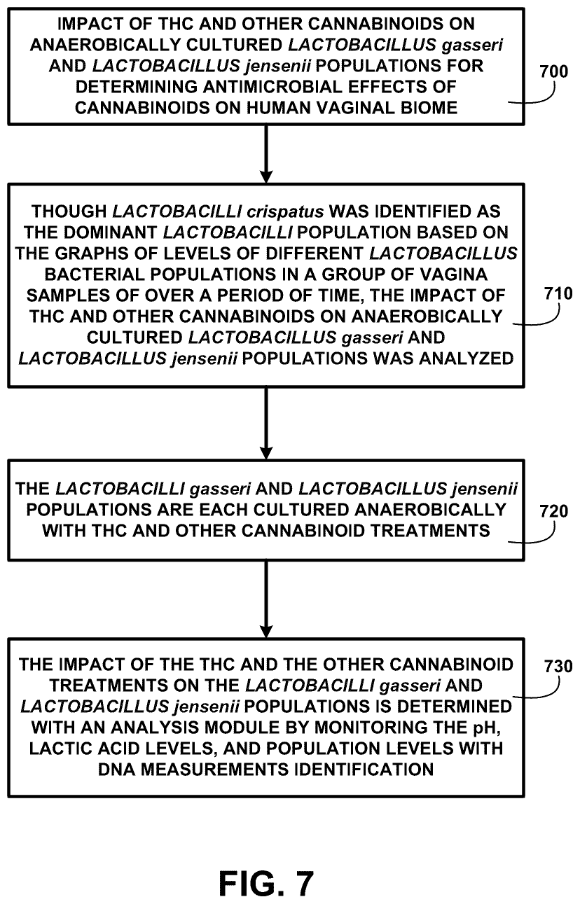

FIG. 7 shows for illustrative purposes only an example of the impact of THC and other cannabinoids on anaerobically cultured Lactobacillus gasseri and Lactobacillus jensenii populations of one embodiment. FIG. 7 shows the impact of THC and other cannabinoids on anaerobically cultured Lactobacillus gasseri and Lactobacillus jensenii populations for determining antimicrobial effects of cannabinoids on human vaginal biome 700. Though Lactobacilli crispatus was identified as the dominant Lactobacilli population based on the graphs of levels of different Lactobacillus bacterial populations in a group of vagina samples over a period of time, the impact of THC and other cannabinoids on anaerobically cultured Lactobacillus gasseri and Lactobacillus jensenii populations was analyzed 710 additionally.

The Lactobacilli gasseri and Lactobacillus jensenii populations are each cultured anaerobically with THC and other cannabinoid treatments 720. The impact of the THC and the other cannabinoid treatments on the Lactobacilli gasseri and Lactobacillus jensenii populations is determined with an analysis module by monitoring the pH, lactic acid levels, and population levels with DNA measurements identification 730 of one embodiment.

### Non-Cannabinoid Parameter Adjustments

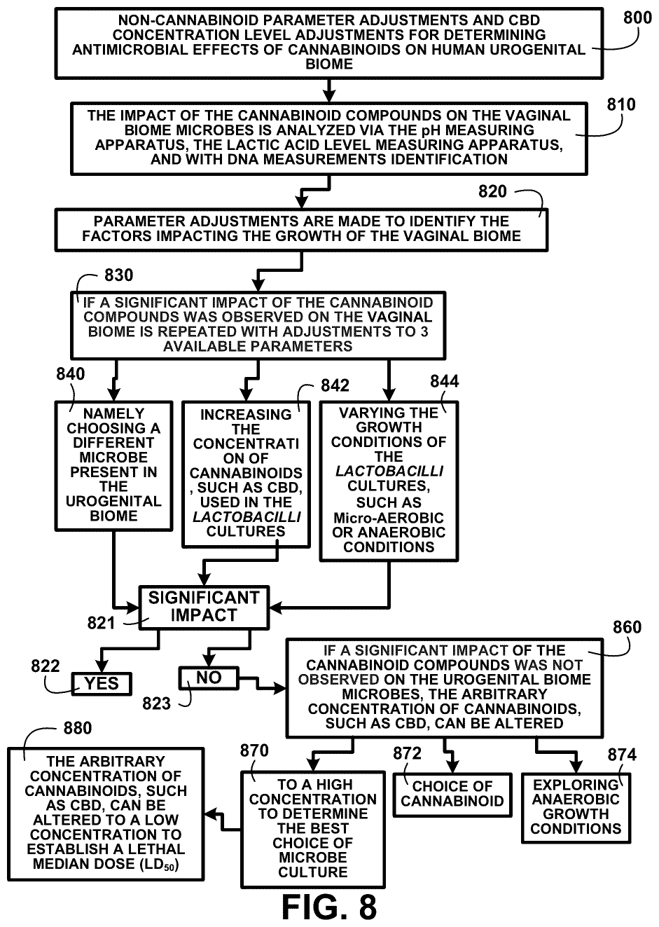

FIG. 8 shows for illustrative purposes only an example of non-cannabinoid parameter adjustments of one embodiment. FIG. 8 shows non-cannabinoid parameter adjustments and CBD concentration level adjustments for determining the antimicrobial effects of cannabinoids on the human vaginal biome 800. The impact of the cannabinoid compounds on the vaginal biome microbes is analyzed via the pH measuring apparatus, the lactic acid level measuring apparatus, and the DNA measurements identification 810.

Parameter adjustments are made to identify the factors impacting the growth of the vaginal biome 820. If a significant impact of the cannabinoid compounds was observed on the vaginal biome is repeated with adjustments to 3 available parameters 830. Namely choosing a different microbe present in the human urogenital microbiome 840, increasing the concentration of cannabinoids, such as CBD, used in the Lactobacilli cultures 842, and varying the growth conditions of the Lactobacilli cultures, such as micro-aerobic or anaerobic conditions 844. Was a significant impact 821 observed Yes 822.

Was a significant impact 821 observed No 823. If a significant impact of the cannabinoid compounds was not observed on the urogenital microbiome microbes, the arbitrary concentration of cannabinoids, such as CBD, can be altered 860 to a high concentration to determine the best choice of microbe culture 870, choice of cannabinoid 872. Exploring anaerobic growth conditions 874, the arbitrary concentration of cannabinoids, such as CBD, can be altered to a low concentration to establish a lethal median dose (LD₅₀) 880 of one embodiment.

### Depicting an Overview of a Cell Culture and Analysis Procedure

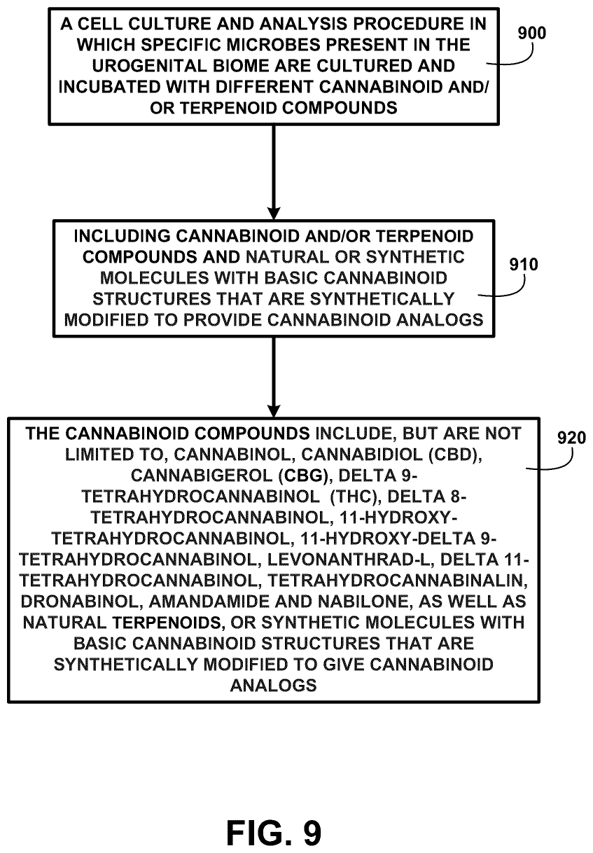

FIG. 9 shows for illustrative purposes only an example of depicting an overview of a cell culture and analysis procedure of one embodiment. FIG. 9 shows a cell culture and analysis procedure in which specific microbes present in the urogenital microbiome are cultured and incubated with different cannabinoid and/or terpenoid compounds 900. Including cannabinoid and/or terpenoid compounds and natural or synthetic molecules with basic cannabinoid structures that are synthetically modified to provide cannabinoid analogs 910. The cannabinoid compounds include, but are not limited to, cannabinol, cannabidiol (CBD), cannabigerol (CBG), delta 9-tetrahydrocannabinol (THC), delta 8-tetrahydrocannabinol, 11-hydroxy-tetrahydrocannabinol, 11-hydroxy-delta 9-tetrahydrocannabinol, levonanthrad-I, delta 11-tetrahydrocannabinol, tetrahydrocannabinalin, dronabinol, anandamide, and nabilone, as well as natural terpenoids, or synthetic molecules with basic cannabinoid structures that are synthetically modified to give cannabinoid analogs 920 of one embodiment.

Terpenoid compounds include, but are not limited to, α-pinene, β-pinene, limonene, β-caryophyllene, eugenol, β-myrcene, γ-terpinolene, geraniol, menthol, α-bisabolol, thymol, humulene, eucalyptol.

### Pure Lactobacillus crispatus Cultures are Grown in a Lactobacillus Selective Media

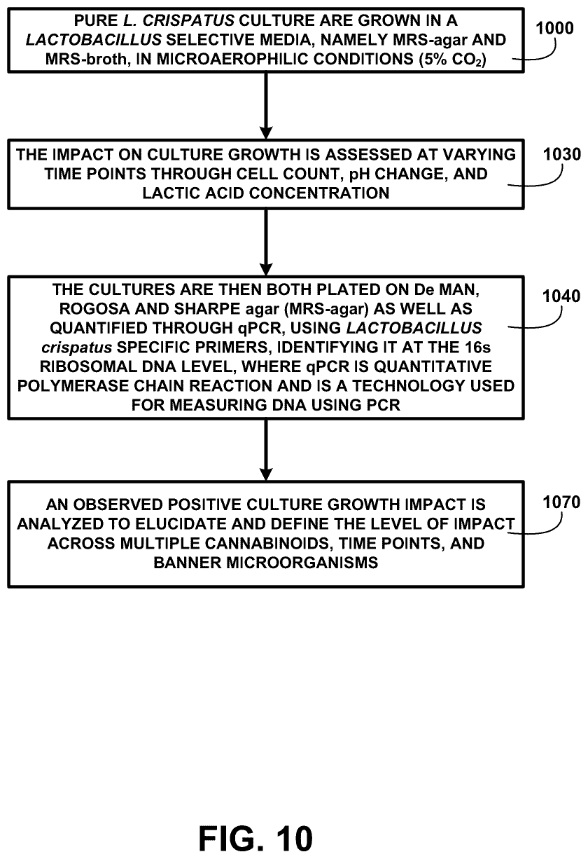

FIG. 10 shows for illustrative purposes only an example of pure Lactobacillus crispatus cultures that are grown in a Lactobacillus selective media of one embodiment. FIG. 10 shows pure Lactobacillus crispatus cultures are grown in a Lactobacillus selective media, namely MRS-agar and MRS-broth, in microaerophilic conditions (5% CO₂) 1000. Cultures are grown at 37° C. for 24-48 hours, or until confluent. This master inoculum is then sub-cultured into fresh broth containing either 100 mg THC, 100 mg CBD, a 50:50 combination mixture of the two, or pure broth as a control. An impact on culture growth is assessed at varying time points through cell count, pH change, and lactic acid concentration 1030. These cultures are then both plated on De Man, Rogosa And Sharpe agar (MRS-agar) as well as quantified through qPCR, using Lactobacillus crispatus specific primers, identifying it at the 16s ribosomal DNA level, where qPCR is quantitative polymerase chain reaction and is a technology used for measuring DNA using PCR 1040, pH is monitored through a pH meter, and lactic acid concentration is monitored through high-performance liquid chromatography (HPLC). An observed positive culture growth impact is analyzed to elucidate and define the level of impact across multiple cannabinoids, time points, and banner microorganisms 1070 of one embodiment.

### Quantitating an Intimate Care Product's Microbiome Impact Results

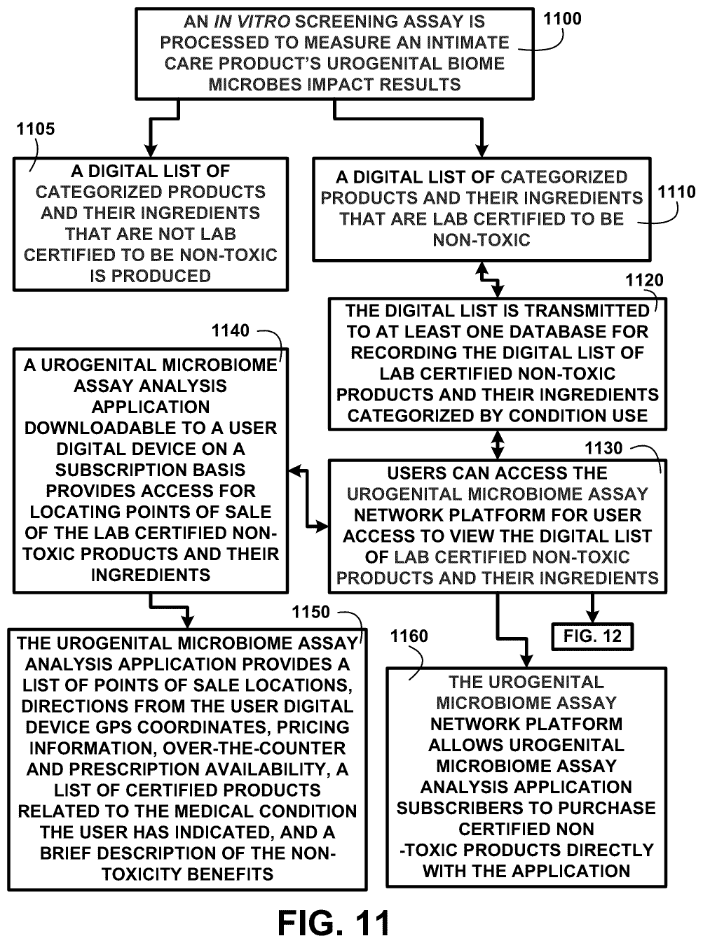

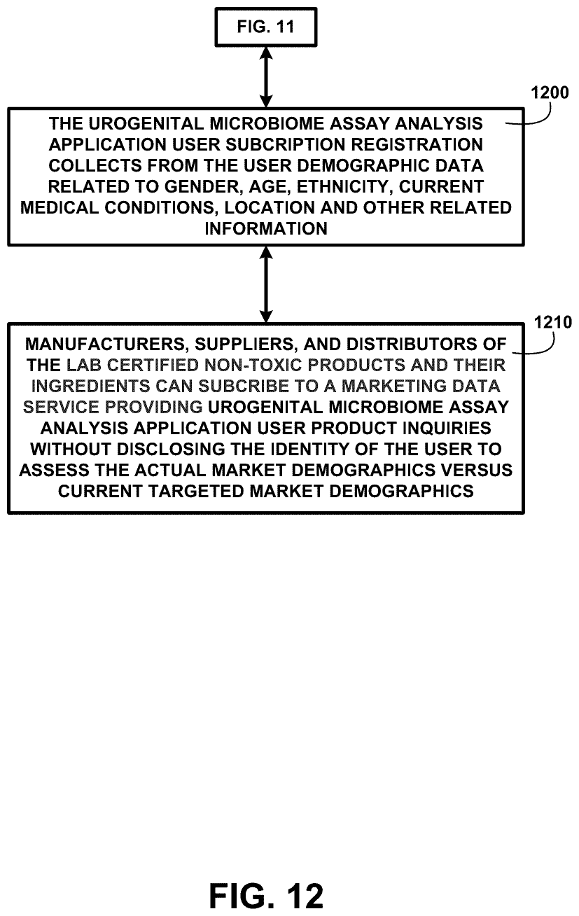

FIG. 11 shows for illustrative purposes only an example of quantitating a personal care product's microbiome impact results of one embodiment. FIG. 11 shows an in vitro screening assay is processed to measure a personal care product's urogenital biome microbe impact results 1100. A digital list of categorized products and their ingredients that are not lab certified to be non-toxic is produced 1105. A digital list of categorized products and their ingredients that are lab certified to be non-toxic 1110 is produced. The digital list is transmitted to at least one database for recording the digital list of lab-certified non-toxic products and their ingredients categorized by condition use 1120. Users can access the urogenital microbiome assay network platform for user access to view the digital list of lab-certified non-toxic products and their ingredients 1130. Descriptions continue on FIG. 12.

A urogenital microbiome assay analysis application downloadable to a user digital device on a subscription basis provides access for locating points of sale of the lab-certified non-toxic products and their ingredients 1140. The urogenital microbiome assay analysis application provides a list of points of sale locations, directions from the user digital device GPS coordinates, pricing information, over-the-counter and prescription availability, a list of certified products related to the medical condition the user has indicated, and a brief description of the non-toxicity benefits 1150. The urogenital microbiome assay network platform allows urogenital microbiome assay analysis application subscribers to purchase certified non-toxic products directly with the application 1160 of one embodiment.

### The Urogenital Microbiome Assay Analysis Application

FIG. 12 shows for illustrative purposes only an example of the urogenital microbiome assay analysis application of one embodiment. FIG. 12 shows the urogenital microbiome assay analysis application user subscription registration collects from the user demographic data related to gender, age, ethnicity, current medical conditions, location, and other related information 1200. Manufacturers, suppliers, and distributors of the lab certified non-toxic products and their ingredients can subscribe to a marketing data service providing urogenital microbiome assay analysis application user product inquiries without disclosing the identity of the user to assess the actual market demographics versus their current targeted market demographics 1210 of one embodiment.

### Adjusting Assay Parameters

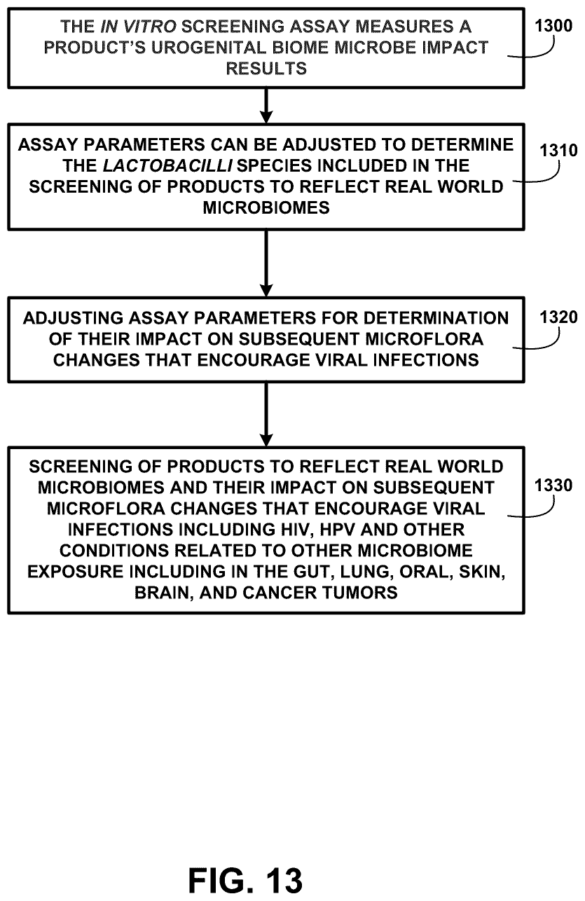

FIG. 13 shows for illustrative purposes only an example of adjusting assay parameters of one embodiment. FIG. 13 shows the in vitro screening assay measures a product's urogenital biome microbe impact results 1300. Assay parameters can be adjusted to determine the lactobacilli species included in the screening of products to reflect real world microbiomes 1310. Adjusting assay parameters for determination of their impact on subsequent microflora changes that encourage viral infections 1320. Screening of products to reflect real world microbiomes and their impact on subsequent microflora changes that encourage viral infections including HIV, HPV. and other conditions related to other microbiome exposure including in the gut, lung, oral, skin, brain, and cancer tumors 1330 of one embodiment.

### In Vitro Screening Assay Product's Toxic Microbiome Impact Results

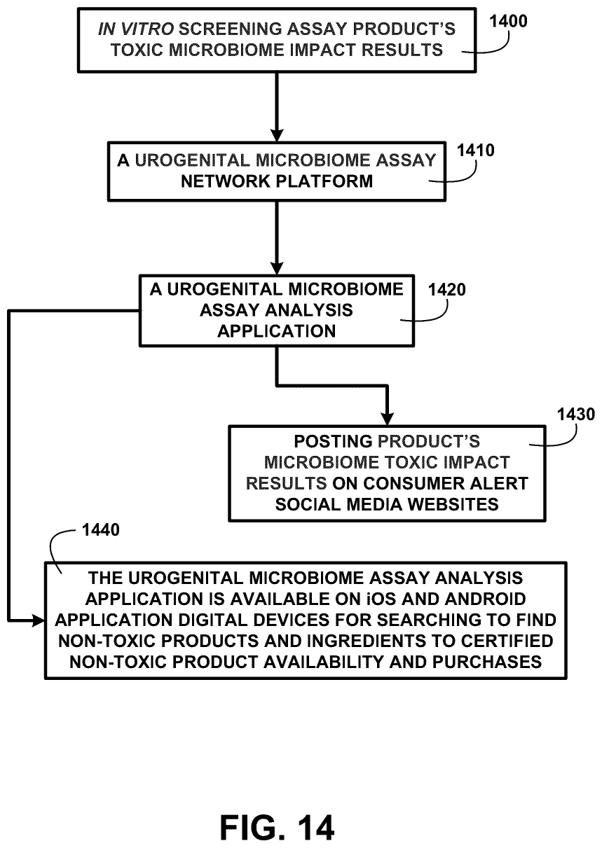

FIG. 14 shows for illustrative purposes only an example of in vitro screening assay product's toxic microbiome impact results of one embodiment. FIG. 14 shows in vitro screening assay product's toxic microbiome impact results 1400 recorded on the urogenital microbiome assay network platform 1410 and accessible with the urogenital microbiome assay analysis application 1420. In one embodiment, posting a product's microbiome toxic impact results on consumer alert social media websites 1430 provides a warning to users that the product they are using might lead to urogenital infections and other undesirable conditions. The urogenital microbiome assay analysis application is available on iOS and Android application digital devices for searching to find non-toxic products and ingredients to certified non-toxic product availability and purchases 1440 of one embodiment.

### The Impact Results for Products Based on the Demographic Differences

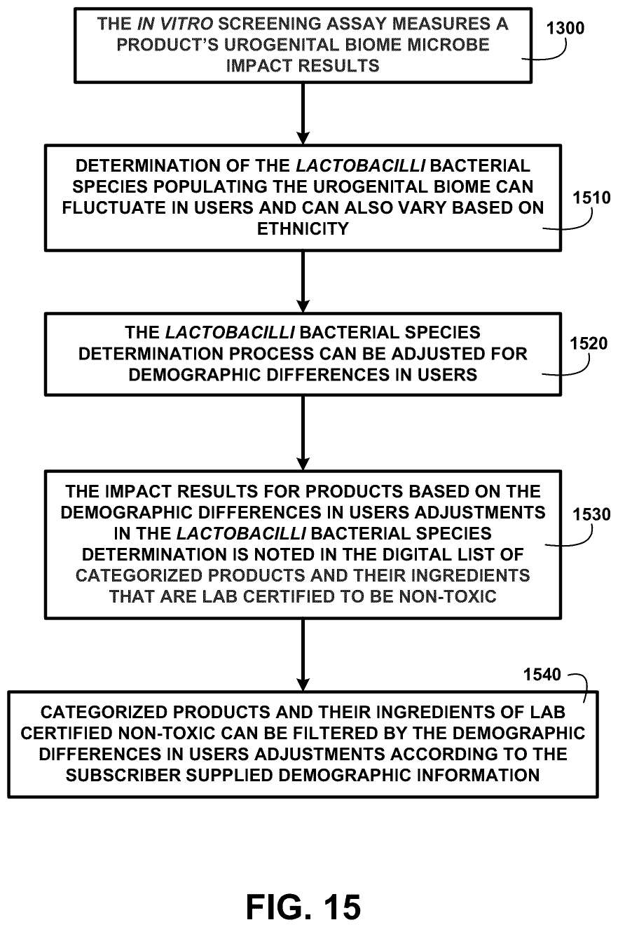

FIG. 15 shows for illustrative purposes only an example of the impact results for products based on the demographic differences of one embodiment. FIG. 15 shows the in vitro screening assay measures a product's urogenital biome microbe impact results 1300. Determination of the lactobacilli bacterial species populating the urogenital biome can fluctuate in users and can also vary based on ethnicity 1510. The lactobacilli bacterial species determination process can be adjusted for demographic differences in users 1520. The impact results for products based on the demographic differences in users adjustments in the lactobacilli bacterial species determination is noted in the digital list of categorized products and their ingredients that are lab certified to be non-toxic 1530. For subscriber users categorized products and their ingredients of lab certified non-toxic can be filtered by the demographic differences in users adjustments according to the subscriber supplied demographic information 1540 of one embodiment.

### Studying CBD and Other Cannabinoids on Anaerobically Cultured Candida albicans

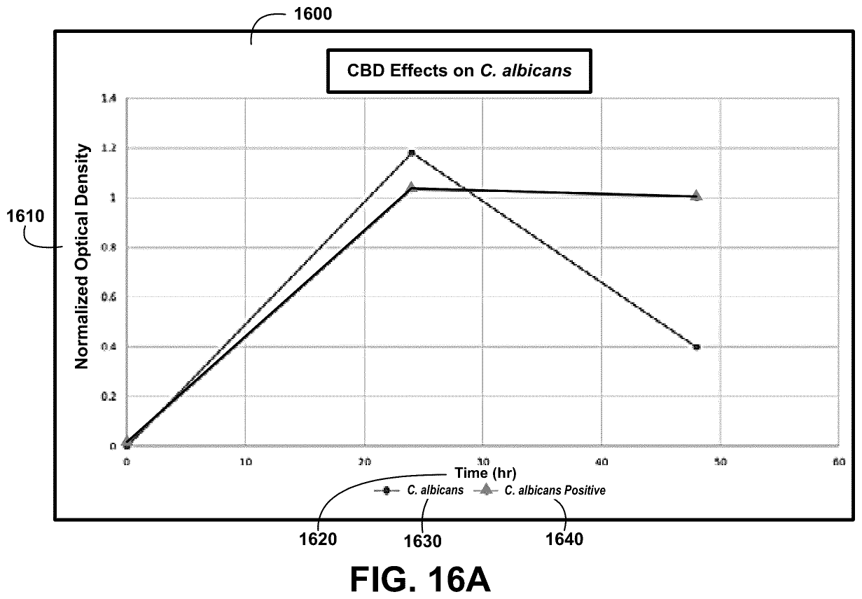

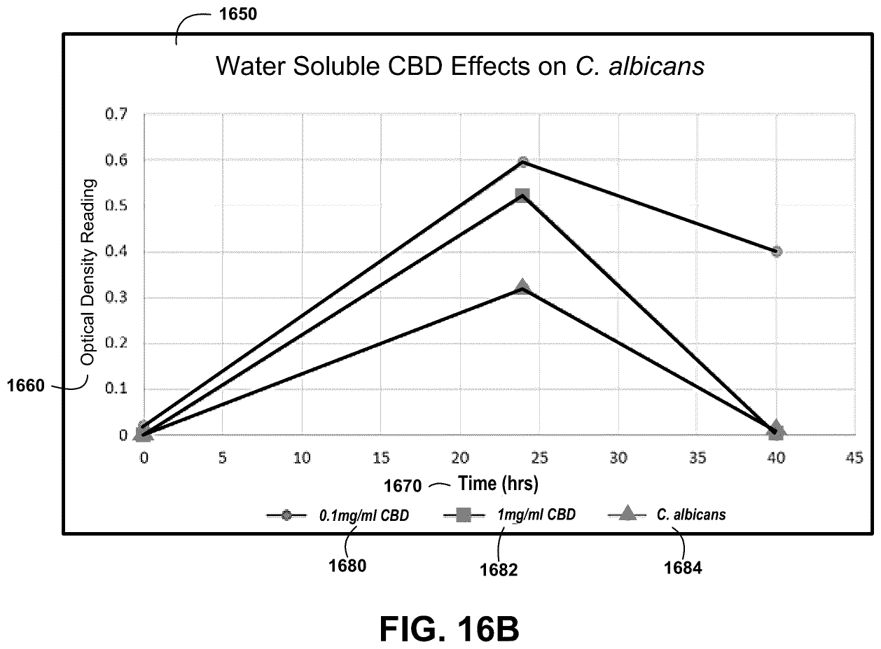

FIG. 16A shows for illustrative purposes only an example of studying CBD and other cannabinoids on anaerobically cultured Candida albicans of one embodiment. FIG. 16A shows a graph of the CBD Effects on C. albicans 1600. The graph includes Normalized Optical Density 1610 on the left Y axis and Time (hr.) 1620 on the bottom X axis. The graphed results show C. albicans 1630 that peak and drop off. The graphed results show C. albicans Positive 1640 that indicate a positive result for CBD effects on the anaerobically cultured Candida albicans.

### Water Soluble CBD Effects on C. albicans

FIG. 16B shows for illustrative purposes only an example of water-soluble CBD effects on C. albicans of one embodiment. FIG. 16B shows a graph of water-soluble CBD effects on C. albicans 1650. The effects are shown with an Optical Density Reading 1660 over intervals of Time (hrs.) 1670. The results indicated with a circle are for a 0.1 mg/ml CBD 1680 treatment, a square for a 1mg/m1CBD 1682 treatment, and a triangle for C. albicans 1684.

The yeast strain (C. albicans) used in this experiment was grown on Sabouraud Dextrose (SD) agar plates under aerobic conditions for 48 hours at 37° C. from frozen glycerin stocks. Individual 10 mL cultures of sterile SD broth were inoculated with single colonies from the plated yeast spp. The inoculated SD culture tubes were placed under aerobic conditions for 48 hours at 37° C. to saturation. For the product dosing, 500 μL was directly added to a labeled 15 ml conical tube containing 9 ml of SD broth, to which 1 ml of a 1:100 yeast dilution was added. Negative controls contained 10 ml of SD broth and 500 μL of product aliquot being tested. An uninoculated sterile SD broth blank was used as well. The treatments were incubated under aerobic conditions at 37° C. Time-points were taken at 0, 24, and 48 hours for optical density (OD) readings using sterile disposable cuvettes each time of one embodiment.

### Quantitative Analysis of Actives for Cannabinoids

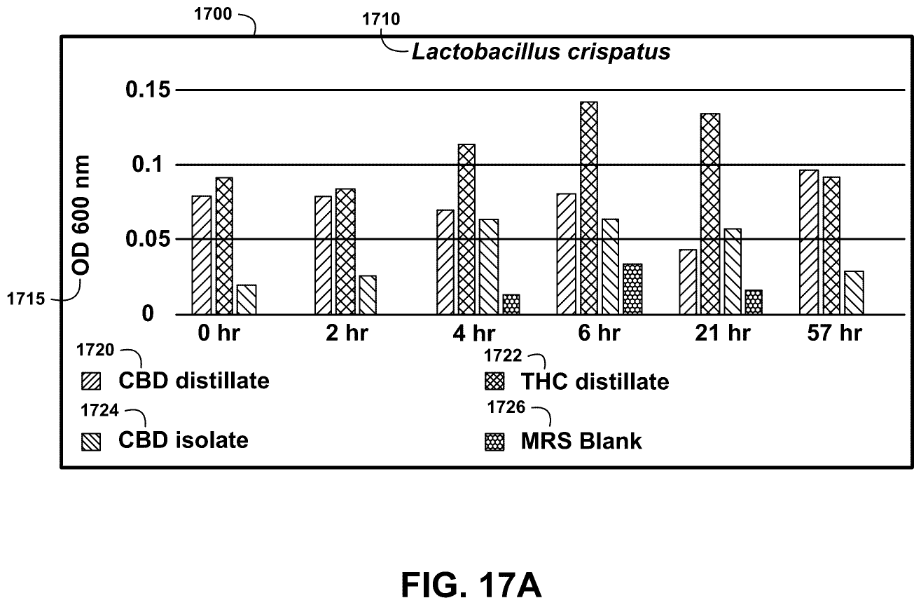

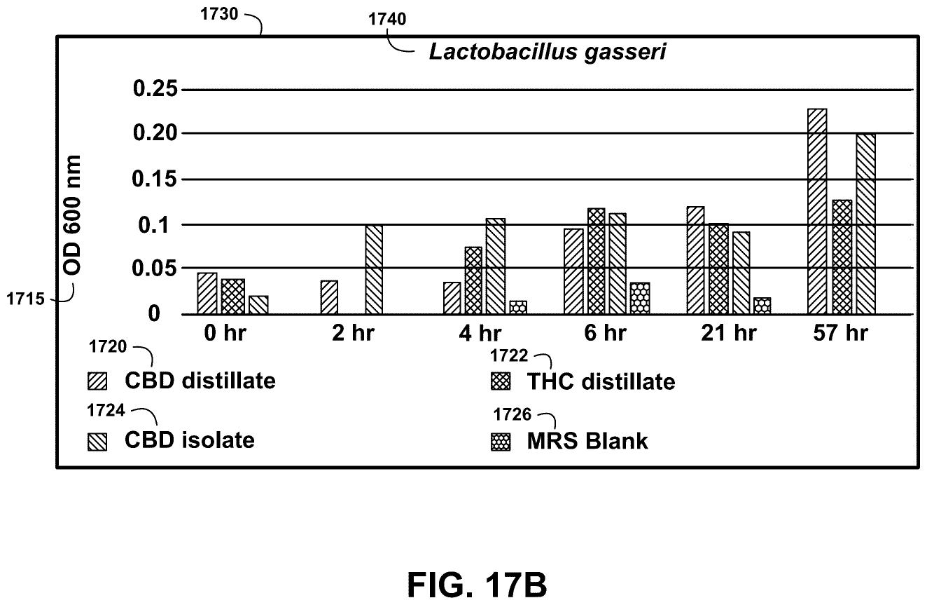

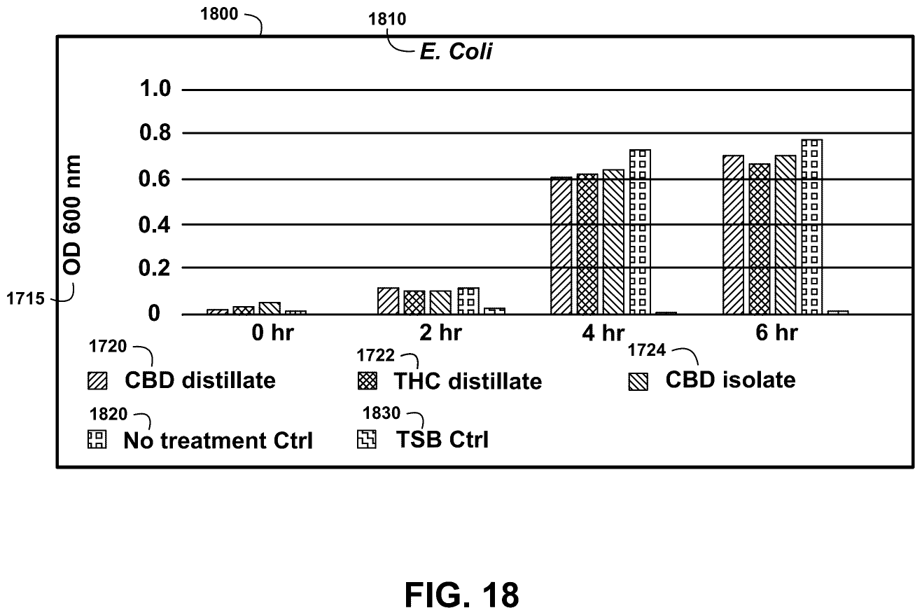

FIGS. 17A, 17B and 18 show the results of quantitative analysis of actives for cannabinoids for Lactobacillus crispatus, Lactobacillus gasseri and E. coli. Each sample is treated with a CBD distillate, THC distillate, and De Man, Rogosa and Sharpe (MRS) agar plates as indicated on chart. Additionally, a CBD isolate of the samples is also treated.

### Results for Lactobacillus crispatus

FIG. 17A shows for illustrative purposes only an example of the results for Lactobacillus crispatus of one embodiment. FIG. 17A shows the quantitative analysis of actives for cannabinoids for Lactobacillus crispatus 1710 in chart 1700 normalized with unexposed culture control data. CBD distillate 1720 treatment results show relatively constant results over treatment exposure times. THC distillate 1722 treatment results show significant results increases over treatment exposure times from 4 hours to 21 hours when decreased results emerge. CBD isolate 1724 treatment results show similar results as the THC distillate 1722 treatment over the same treatment exposure times. The MRS blank 1726 treatment show no result until the 4 hr. exposure time with an increase in the 6 hr. exposure time when it decreases into the 21 hr. exposure time and then no results following. The treatment results are measured by OD 600 nm 1715 and the result chart bars reflect the measurement at the various exposure times of one embodiment.

### Results for Lactobacillus gasseri

FIG. 17B shows for illustrative purposes only an example of the results for Lactobacillus gasseri of one embodiment. FIG. 17B shows the quantitative analysis of actives for cannabinoids for Lactobacillus gasseri 1740 in chart 1730 normalized with unexposed culture control data. CBD distillate 1720 treatment results show relatively constant increased results over treatment exposure times until the 57 hr. time when the results show an increased spike in the results. THC distillate 1722 treatment results show fluctuating results with increases and decreases over treatment exposure times. CBD isolate 1724 treatment results show similar increased results as the CBD distillate 1720 treatment over the same treatment exposure times. The MRS blank 1726 treatment show no result until the 4 hr. exposure time with an increase in the 6 hr. exposure time when it decreases into the 21 hr. exposure time and then no results following. The treatment results are measured by OD 600 nm 1715 and the result chart bars reflect the measurement at the various exposure times of one embodiment.

### Results for E. Coli

FIG. 18 shows for illustrative purposes only an example of the results for E. Coli of one embodiment. FIG. 18 shows the quantitative analysis of actives for cannabinoids for E. coli 1810 in chart 1800. CBD distillate 1720, THC distillate 1722, and CBD isolate 1724 treatment results show relatively similar results over treatment exposure times as the No treatment Ctrl 1820 wherein the results show a significant increase spike in the 4 hr. exposure time continuing in through the 6 hr. The TSB Ctrl 1830 shows insignificant measurements from the 2 hr. to 6 hr. treatment exposure times.

The charts illustrate that lactobacilli does not flourish under aerobic conditions at 30° C. which may influence the outcome of the drug exposure. While E. coli demonstrated classic growth with no inhibition by any drug treatment under aerobic conditions. The treatment results are measured by OD 600 nm 1715 of one embodiment.

### L. crispatus in Anaerobic Conditions at 37° C.

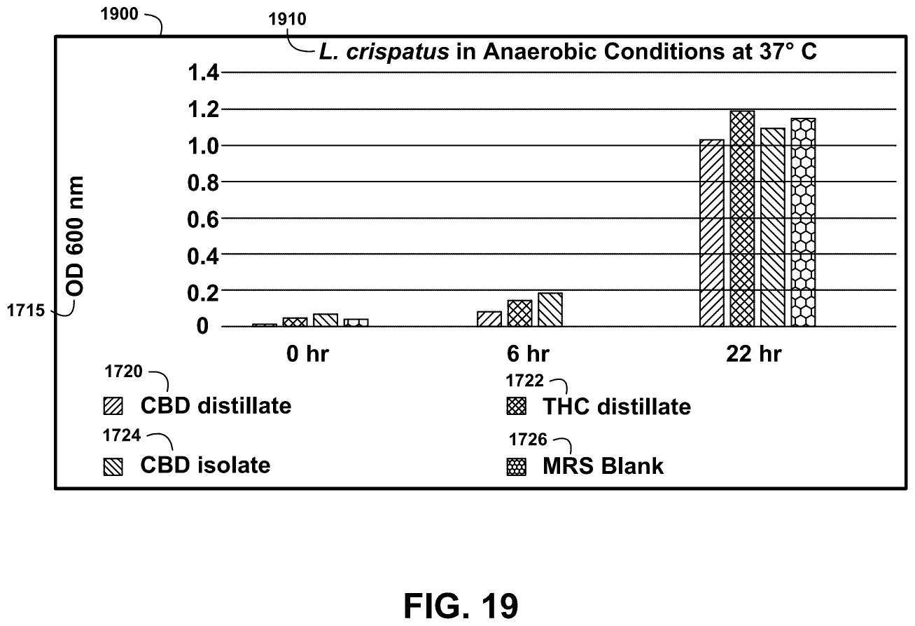

FIG. 19 shows for illustrative purposes only an example of L. crispatus in Anaerobic Conditions at 37° C. of one embodiment. FIG. 19 shows a chart 1900 of treatment results for L. crispatus in Anaerobic Conditions at 37° C. 1910. In comparison to growth under aerobic conditions, exposure of the same two lactobacilli cultures under anaerobic conditions at 37° C., demonstrated lack of inhibition for treatment results over treatment exposure times by CBD distillate 1720, THC distillate 1722, CBD isolate 1724, and MRS blank 1726 dosing with classic observations. Confirmation as lactobacilli was confirmed by qPCR. The treatment results are measured by OD 600 nm 1715 of one embodiment.

### L. gasseri in Anaerobic Conditions at 37° C.

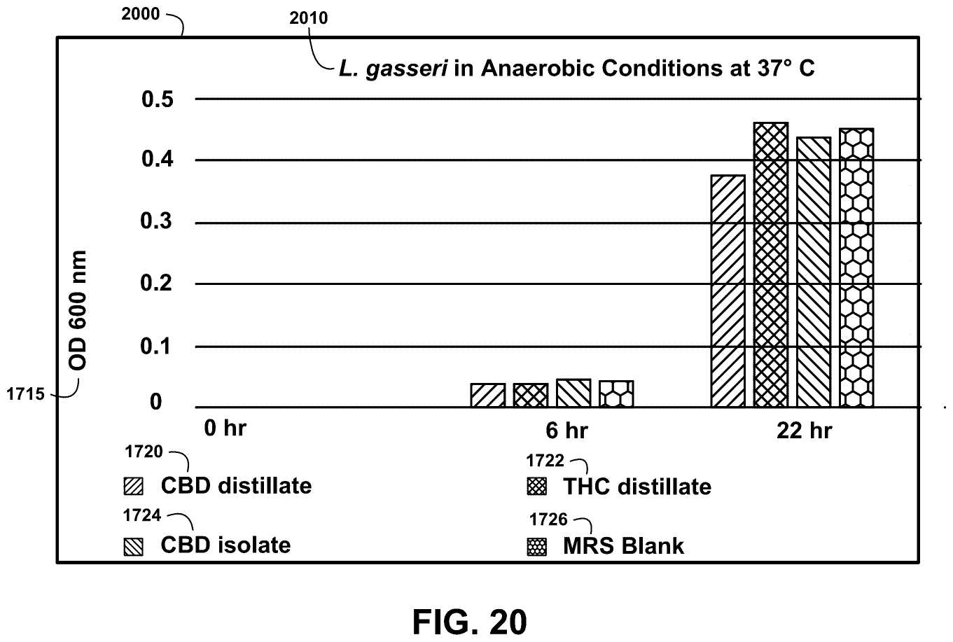

FIG. 20 shows for illustrative purposes only an example of L. gasseri in anaerobic conditions at 37° C. of one embodiment. FIG. 20 shows a chart 2000 of treatment results for L. gasseri in anaerobic conditions at 37° C. 2010. In comparison to growth under aerobic conditions, exposure of the same two lactobacilli cultures under anaerobic conditions at 37° C., demonstrated lack of inhibition for treatment results over treatment exposure times by CBD distillate 1720, THC distillate 1722, CBD isolate 1724, and MRS blank 1726 dosing with classic observations. Confirmation as lactobacilli was confirmed by qPCR. The treatment results are measured by OD 600 nm 1715 of one embodiment.

### Inhibition Results

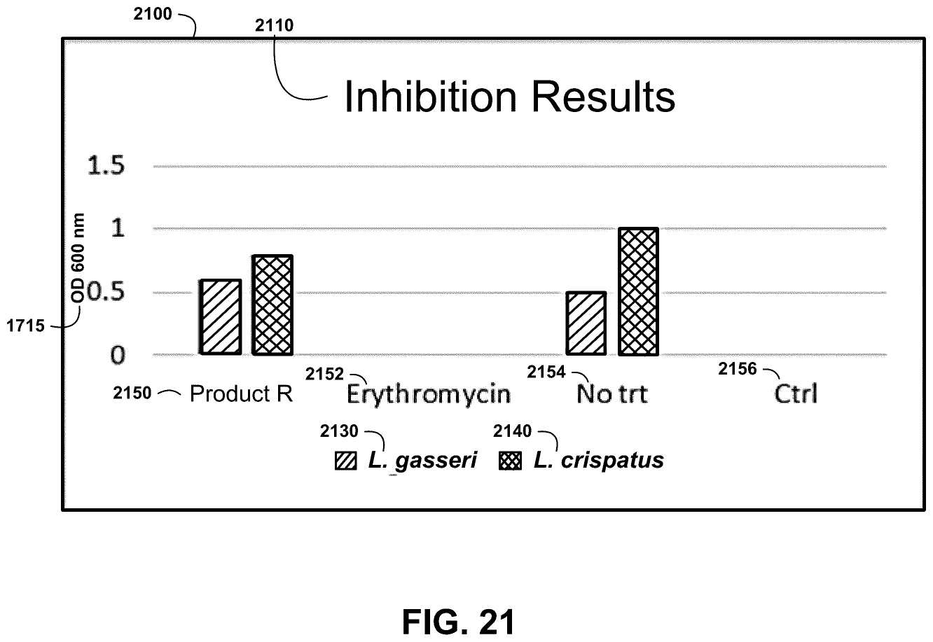

FIG. 21 shows for illustrative purposes only an example of inhibition results of one embodiment. FIG. 21 shows a chart 2100 of inhibition results 2110 lactobacilli strains L. gasseri 2130 and L. crispatus 2140. Treatments included Product R 2150, Erythromycin 2152, No trt 2154, and Ctrl 2156. As expected, there was no observed inhibition of growth under any treatments including the negative control showed no growth in the uninoculated MRS broth. A drop in pH was expected and observed. Follow up treatments should include a positive control using a gram-positive antibiotic, e.g erythromycin, vancomycin or tetracycline, to show inhibition of the lactobacilli strains L. gasseri 2130 and L. crispatus 2140. The treatment results are measured by OD 600 nm 1715 of one embodiment.

### L. gasseri in Anaerobic Conditions at 30° C.

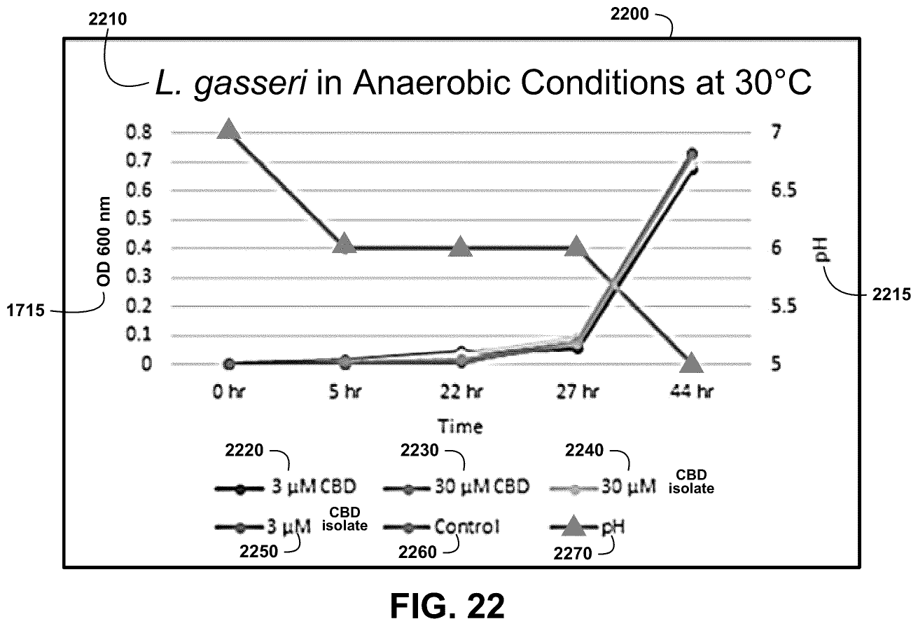

FIG. 22 shows for illustrative purposes only an example of L. gasseri in anaerobic conditions at 30° C. of one embodiment. FIG. 22 shows treatment results in chart 2200 of L. gasseri in anaerobic conditions at 30° C. 2210. This treatment includes pH 2215 monitoring, and the treatment results are measured by OD 600 nm 1715. Because inhibitory levels of CBD against other gram-positive microorganisms e.g., Staphylococcus aureus and Streptococcus pneumoniae have been reported in the low micromolar range, a lower dose response experiment was set up for the CBD distillate 1720 of FIG. 17A and the CBD isolate 1724 of FIG. 17A. The treatment results are shown for 3 μM CBD 2220, 30 μM CBD 2230, 30 μM CBD isolate 2240, 3 μM CBD isolate 2250, Control 2260, and pH 2270. In this inhibition chart it shows pH changes in the bacterial cultures were also monitored since the presumed mechanism by which lactobacilli creates a beneficial vaginal environment is through maintenance of pH levels near 4 which in and of itself is inhibitory to many other invasive bacteria and viruses of one embodiment.

### Cultures of Lactobacilli jensenii Exposed to Three Different CBD Products

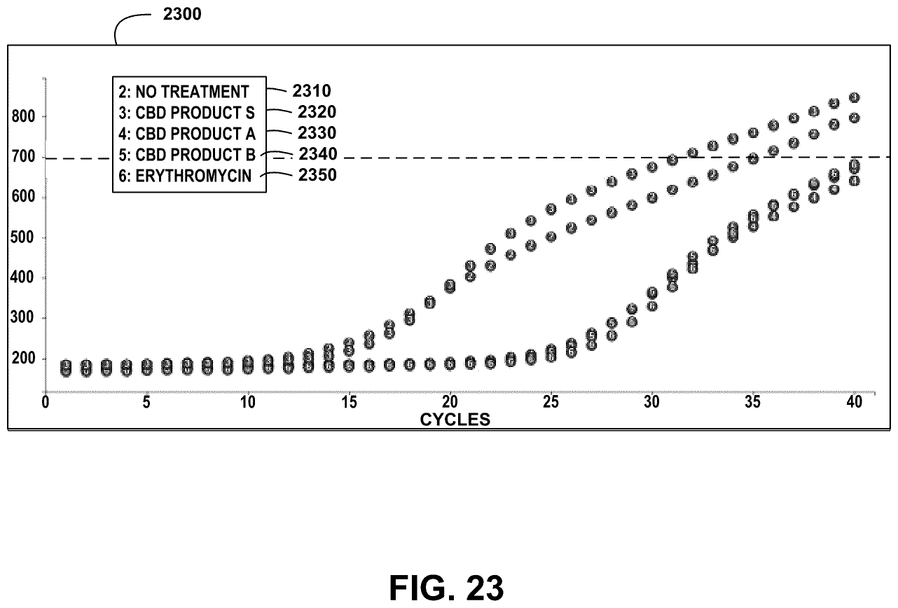

FIG. 23 shows for illustrative purposes only an example of results of cultures of Lactobacilli jensenii exposed to three different CBD products of one embodiment. FIG. 23 shows the results of cultures of Lactobacilli jensenii exposed to three different CBD products 2300. The three different CBD products include CBD product S 2320, CBD product A 2330, and CBD product B 2340. The cultures included a no treatment 2310 culture, and an Erythromycin 2350 culture. The cultures of Lactobacilli jensenii exposed to three different CBD products were performed at [10 mg/m L] for 24 hours under anaerobic conditions. Culture samples were evaluated via qPCR reagents.

The dashed line indicates point of inhibition as per antibiotic control at [0.2 mg/mL]. CBD product S was not inhibitory while CBD products A and B were inhibitory. The graphed summation of the cultures results assay illustrates the variations in the results based on the three different CBD products of one embodiment.

### Commercial Products Tested Results

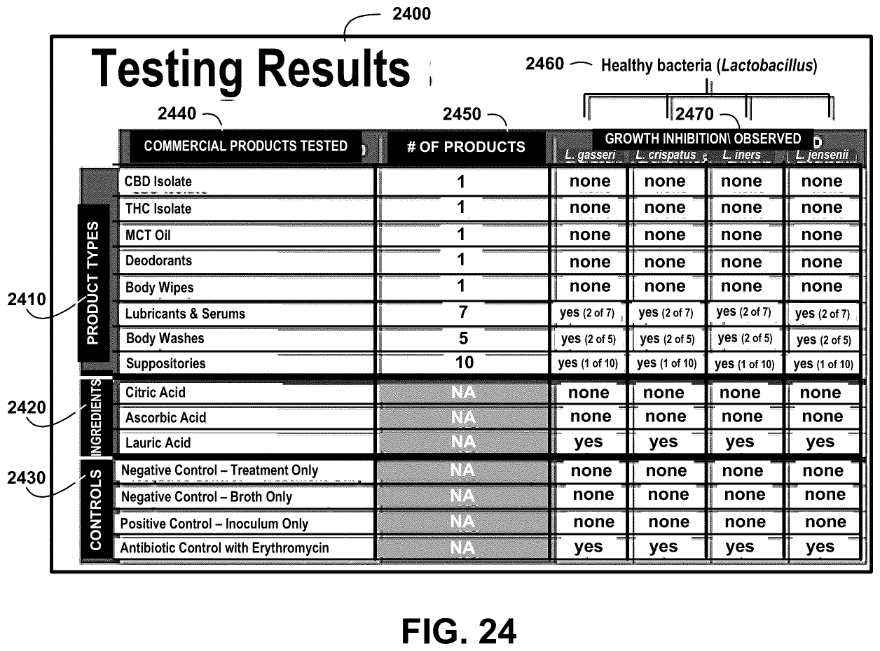

FIG. 24 shows for illustrative purposes only an example of commercial products tested results of one embodiment. FIG. 24 shows testing results 2400 of commercial products tested 2440 on a number of products 2450. The testing of commercial products tested 2440 on a number of products 2450 is for checking for growth inhibition observed 2470 in healthy bacteria (Lactobacillus) 2460. The testing included various product types 2410 with different ingredients 2420. The testing protocol included a number of controls 2430 of one embodiment.

### Normalized CT Values to Control

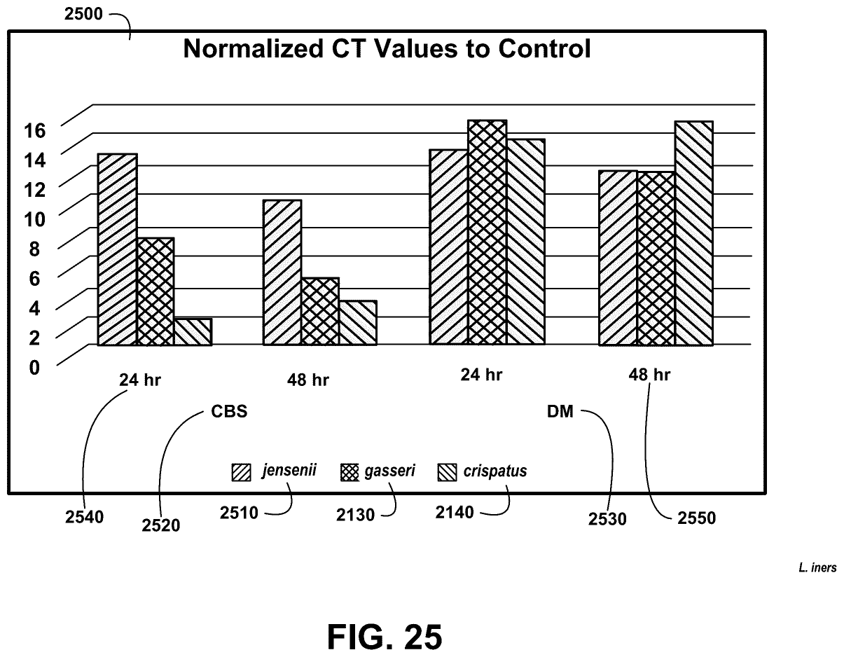

FIG. 25 shows for illustrative purposes only an example of normalized CT values to control of one embodiment. FIG. 25 shows a bar chart of Normalized CT Values to Control 2500. The culture samples included jensenii 2510, gasseri 2130, and crispatus 2140. The CBS 2520 values cover culture times of 24 hr. 2540 and 48 hr. 2550. The DM 2530 values also cover culture times of 24 hr. 2540 and 48 hr. 2550 of one embodiment.

### Control Normalized at log10 CFU/MI

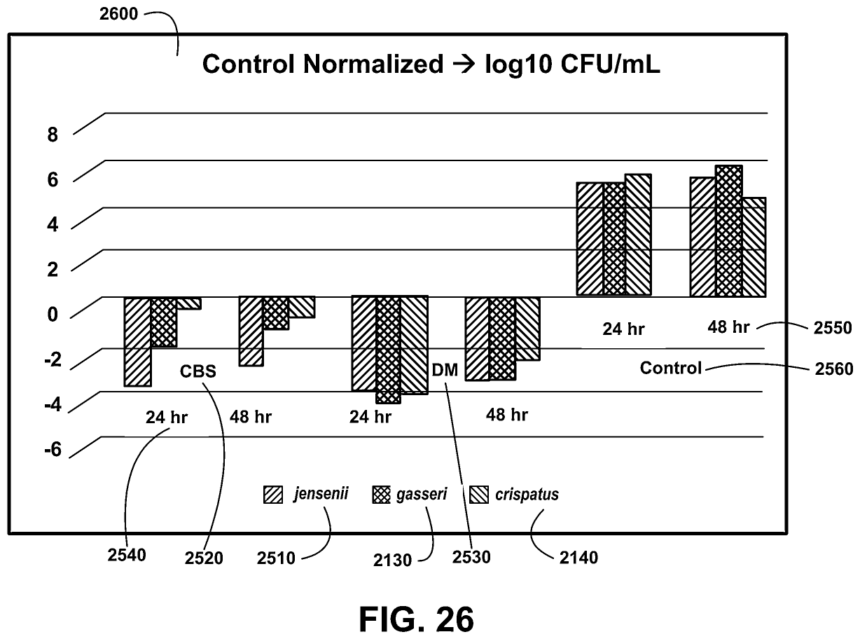

FIG. 26 shows for illustrative purposes only an example of control normalized at log10 CFU/mL of one embodiment. FIG. 26 shows a bar chart of Control Normalized at log10 CFU/mL 2600. The culture samples included jensenii 2510, gasseri 2130, and crispatus 2140. The CBS 2520 values cover culture times of 24 hr. 2540 and 48 hr. 2550. The DM 2530 values also cover culture times of 24 hr. 2540 and 48 hr. 2550. The Control 2560 values also cover culture times of 24 hr. 2540 and 48 hr. 2550 of one embodiment.

### Exposure to CBD Product A

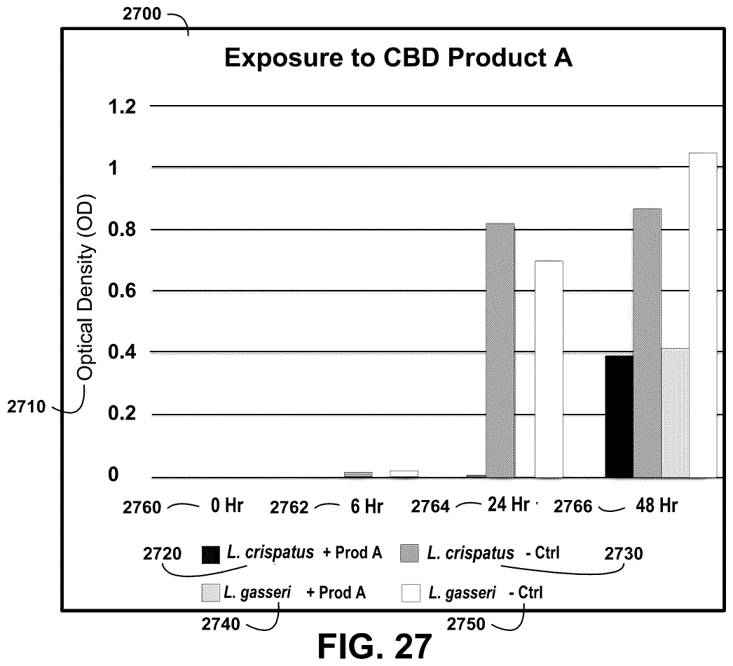

FIG. 27 shows for illustrative purposes only an example of Exposure to CBD Product A of one embodiment. FIG. 27 shows a bar chart of exposure to CBD product A 2700 of individual species of urogenital lactobacilli. The results of the exposure are measured with Optical Density (OD) 2710 devices. In this embodiment the exposed species of urogenital lactobacilli include L. crispatus+Prod A 2720, L. crispatus+Ctrl 2730, L. gasseri+Prod A 2740, and L. gasseri+Ctrl 2750. The results are shown for exposure times of 0 Hr. 2760, 6 Hr. 2762, 24 Hr. 2764, and 48 Hr. 2766 of one embodiment.

### Exposure to CBD/THC Product B

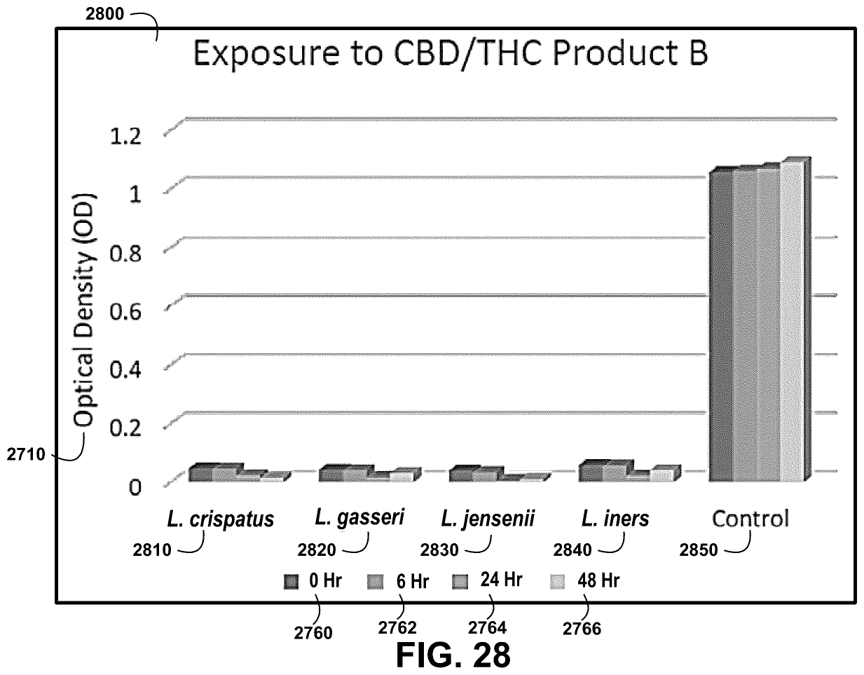

FIG. 28 shows for illustrative purposes only an example of Exposure to CBD/THC Product B of one embodiment. FIG. 28 shows a bar chart of exposure to CBD/THC Product B 2800. The results of the exposures are measured using Optical Density (OD) 2710 devices. Selected species of urogenital lactobacilli for exposure include L. crispatus 2810, L. gasseri 2820, L. jensenii 2830, L. iners 2840, and a Control 2850. The results vary with the exposure times of 0 Hr. 2760, 6 Hr. 2762, 24 Hr. 2764, and 48 Hr. 2766 of one embodiment.

### Body Wash Exposure Study

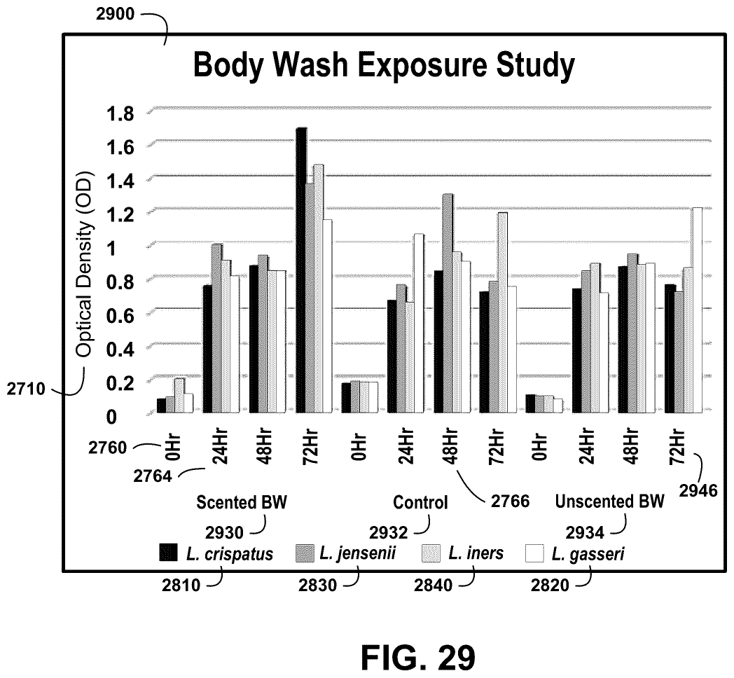

FIG. 29 shows for illustrative purposes only an example of a body wash exposure study of one embodiment. FIG. 29 shows a bar chart of a Body Wash Exposure Study 2900. The results of the exposures are measured using Optical Density (OD) 2710 devices. The Body Wash Exposure Study 2900 is performed on selected individual species of urogenital lactobacilli. The selected individual species of urogenital lactobacilli include L. crispatus 2810, L. gasseri 2820, L. jensenii 2830, and L. iners 2840. The Body Wash Exposure Study 2900 is performed with body wash (BW) that includes Scented BW 2930 and Unscented BW 2934 and includes a Control 2932. The exposure times are intervals including 0 Hr. 2760, 24 Hr. 2764, 48 Hr. 2766, and 72 Hr. 2946 of one embodiment.

The foregoing has described the principles, embodiments and modes of operation of the present invention. However, the invention should not be construed as being limited to the particular embodiments discussed. The above-described embodiments should be regarded as illustrative rather than restrictive, and it should be appreciated that variations may be made in those embodiments by workers skilled in the art without departing from the scope of the present invention as defined by the following claims.

1-20. (canceled)

21. A testing system for urogenital products, comprising:
a quantitative polymerase chain reaction analyzer configured to receive a female vaginal test sample with demographic information, to determine and identify DNA identification and quantification information of Lactobacillus bacteria populations in the female test sample and to determine baseline populations of the Lactobacillus bacteria populations;
a treatment compound configured to be exposed to the Lactobacillus bacteria populations;
a microbiome assay network platform coupled to the quantitative polymerase chain reaction analyzer configured to receive the DNA identification and the quantification information and the baseline populations and further configured to track growth inhibition patterns of the Lactobacillus bacteria populations from the baseline populations based on the exposure of the Lactobacillus bacteria to the treatment compound;
a processor coupled to the microbiome assay network platform configured to analyze the growth inhibition patterns of the Lactobacillus bacteria populations from the baseline populations and to rank each growth inhibition pattern for at least one of a beneficial or harmful microorganism based the DNA identification and the demographic information; and
a mobile application coupled to the microbiome assay network platform configured to compare the analyzed and the ranked growth inhibition patterns for the treatment compound and provide an effectiveness rank of the treatment compound.

22. The testing system for urogenital products of claim 21, wherein the treatment compound includes ingredients from a group of at least one natural terpenoids, or synthetic molecules with basic cannabinoid structures including α-pinene, β-pinene, limonene, β-caryophyllene, eugenol, β-myrcene, γ-terpinolene, geraniol, menthol, α-bisabolol, thymol, humulene, eucalyptol.

23. The testing system for urogenital products of claim 21, wherein the treatment compound is a cannabinoid treatment compound with ingredients from a group of at least one of cannabinol, cannabidiol (CBD), cannabigerol (CBG), delta 9-tetrahydrocannabinol (THC), delta 8-tetrahydrocannabinol, 11-hydroxy-tetrahydrocannabinol, 11-hydroxy-delta 9-tetrahydrocannabinol, levonanthrad-I, delta 11-tetrahydrocannabinol, tetrahydrocannabinalin, dronabinol, anandamide, and nabilone.

24. The testing system for urogenital products of claim 21, wherein the treatment compound is a cannabinoid treatment compound having a range of 1 mg to 10 mg of cannabidiol (CBD).

25. The testing system for urogenital products of claim 21, wherein the treatment compound is a cannabinoid treatment compound having cannabidiol (CBD).

26. The testing system for urogenital products of claim 21, wherein the treatment compound is a cannabinoid treatment compound having a range of 1 mg to 10 mg Tetrahydrocannabinol (THC).

27. The testing system for urogenital products of claim 21, wherein the treatment compound is a cannabinoid treatment compound having a 50:50 ratio of cannabidiol (CBD) and Tetrahydrocannabinol (THC).

28. The testing system for urogenital products of claim 21, wherein the quantitative polymerase chain reaction analyzer is further configured to determine qualitative information of microorganisms testing results to derive at least one of an inhibitory or non-inhibitory outcomes information about the Lactobacillus bacteria populations.

29. The testing system for urogenital products of claim 21, wherein the quantitative polymerase chain reaction analyzer is further configured to determine the existence of Lactobacillus crispatus, Lactobacillus gasseri, and Lactobacillus jensenii populations.

30. The testing system for urogenital products of claim 21, wherein the microbiome assay network platform is further configured to receive the DNA identification and the quantification information and the baseline populations and further configured to analyze antifungal growth patterns of the female test sample when exposed to the treatment compound.

31. A testing system for urogenital products, comprising:
a quantitative polymerase chain reaction analyzer configured to receive a female vaginal test sample with demographic information, to determine and identify DNA identification and quantification information of Lactobacillus bacteria populations in the female test sample and to determine baseline populations of the Lactobacillus bacteria populations;
a liquid chromatography device coupled to the quantitative polymerase chain reaction analyzer configured to measure and record a lactic acid concentration in the female test sample;
a pH measuring apparatus coupled to the quantitative polymerase chain reaction analyzer configured to measure and record a pH level in the female test sample;
a treatment compound configured to be exposed to the Lactobacillus bacteria populations;
a microbiome assay network platform coupled to the quantitative polymerase chain reaction analyzer configured to receive the DNA identification and the quantification information, the lactic acid concentration, the pH level and the baseline populations and further configured to track growth inhibition patterns of the Lactobacillus bacteria populations from the baseline populations based on the exposure of the Lactobacillus bacteria to the treatment compound;
a processor coupled to the microbiome assay network platform configured to analyze the growth inhibition patterns of the Lactobacillus bacteria populations from the baseline populations and to rank each growth inhibition pattern for at least one of a beneficial or harmful microorganism based the DNA identification; and
a mobile application coupled to the microbiome assay network platform configured to compare the analyzed and the ranked growth inhibition patterns for the treatment compound and provide an effectiveness rank of the treatment compound.

32. The testing system for urogenital products of claim 31, wherein the treatment compound includes ingredients from a group of at least one natural terpenoids, or synthetic molecules with basic cannabinoid structures including α-pinene, β-pinene, limonene, β-caryophyllene, eugenol, β-myrcene, γ-terpinolene, geraniol, menthol, α-bisabolol, thymol, humulene, eucalyptol.

33. The testing system for urogenital products of claim 31, wherein the treatment compound is a cannabinoid treatment compound with ingredients from a group of at least one of cannabinol, cannabidiol (CBD), cannabigerol (CBG), delta 9-tetrahydrocannabinol (THC), delta 8-tetrahydrocannabinol, 11-hydroxy-tetrahydrocannabinol, 11-hydroxy- delta 9-tetrahydrocannabinol, levonanthrad-I, delta 11-tetrahydrocannabinol, tetrahydrocannabinalin, dronabinol, anandamide, and nabilone.

34. The testing system for urogenital products of claim 31, wherein the treatment compound is a cannabinoid treatment compound having a range of 1 mg to 10 mg of cannabidiol (CBD).

35. The testing system for urogenital products of claim 31, wherein the treatment compound is a cannabinoid treatment compound having cannabidiol (CBD).

36. The testing system for urogenital products of claim 31, wherein the treatment compound is a cannabinoid treatment compound having a range of 1 mg to 10 mg Tetrahydrocannabinol (THC).

37. The testing system for urogenital products of claim 31, wherein the treatment compound is a cannabinoid treatment compound having a 50:50 ratio of cannabidiol (CBD) and Tetrahydrocannabinol (THC).

38. A testing system for urogenital products, comprising:
a quantitative polymerase chain reaction analyzer configured to receive a female vaginal test sample with demographic information, to determine and identify DNA identification and quantification information of Lactobacillus bacteria populations in the female test sample and to determine baseline populations of the Lactobacillus bacteria populations;
a liquid chromatography device coupled to the quantitative polymerase chain reaction analyzer configured to measure and record a lactic acid concentration in the female test sample;
a pH measuring apparatus coupled to the quantitative polymerase chain reaction analyzer configured to measure and record a pH level in the female test sample;
a plurality of treatment compounds, wherein each treatment compound is configured to be exposed individually to the Lactobacillus bacteria populations;
a microbiome assay network platform coupled to the quantitative polymerase chain reaction analyzer configured to receive the DNA identification and the quantification information, the lactic acid concentration, the pH level and the baseline populations and further configured to track growth inhibition patterns of the Lactobacillus bacteria populations from the baseline populations based on the exposure of the Lactobacillus bacteria by each treatment compound;
a processor coupled to the microbiome assay network platform configured to analyze the growth inhibition patterns of the Lactobacillus bacteria populations from the baseline populations based on the exposure of the Lactobacillus bacteria by each treatment compound and to rank each growth inhibition pattern for at least one of a beneficial or harmful microorganism based the DNA identification based on the exposure of the Lactobacillus bacteria by each treatment compound; and
a mobile application coupled to the microbiome assay network platform configured to compare the analyzed and the ranked growth inhibition patterns for each treatment compound and provide an effectiveness rank of each treatment compound.

39. The testing system for urogenital products of claim 38, wherein the treatment compound is a cannabinoid treatment compound having cannabidiol (CBD).

40. The testing system for urogenital products of claim 38, wherein the treatment compound is a cannabinoid treatment compound having a range of 1 mg to 10 mg Tetrahydrocannabinol (THC).
